(19) United States
(12) Patent Application Publication
(10) Pub. No.: US 2005/0281746 A1
(43) Pub. Date: Dec. 22, 2005

(54) Anticoagulant contrast media

(76) Inventor: Laura J. Melton, Durham, NC (US)

Correspondence Address: COOLEY GODWARD, LLP, 3000 EL CAMINO REAL, 5 PALO ALTO SQUARE, PALO ALTO, CA, 94306, US

(21) Appl. No.: 11/153,254

(22) Filed: Jun. 14, 2005

Related U.S. Application Data
(60) Provisional application No. 60/580,624, filed on Jun. 16, 2004.

(51) Int. Cl. A61K 49/00

(52) U.S. Cl. 424/9.34; 530/383

(57) ABSTRACT

The present invention provides novel anticoagulant contrast agents, which comprise an organic scaffolding moiety, an organic anticoagulant moiety, and an imaging moiety. The invention also provides anticoagulant contrast media and methods of visualizing internal structures utilizing the novel anticoagulant contrast agents and anticoagulant contrast media.

### I. CROSS REFERENCE To RELATED APPLICATIONS

This application claims the benefit of U.S. Provisional Application No. 60/580,624, entitled “Anticoagulant Contrast Media” filed on Jun. 16, 2004, the disclosure of which is incorporated herein by reference in its entirety.

### II. INTRODUCTION

A. Field of the Invention

The present invention relates to compounds and compositions for use in diagnostic imaging.

B. Background of the Invention

Cardiovascular disease is the number one cause of death in the United State afflicting approximately 62 million Americans and killing more than 900,000 every year. The most cardiovascular disease-prone segment of the population (adults aged 55 years or older) has been growing twice as fast as the United States population as a whole, at about 1.8% per year. However, the growth of the disease-prone segments alone does not account for the 4%-8% increase in most types of heart surgeries.

Nearly 2 million hospital administrations per year are based on a diagnosis of acute coronary syndrome (ACS). The clinical expression of coronary artery disease is driven by a series of pathobiologic events that include plaque disruption and varying degrees of intravascular thromboembolism. Rupture of an atherosclerotic plaque with subsequent thrombosis due to platelet aggregation and activation of the intrinsic and extrinsic pathways of coagulation are the initiating events in patients with ACS. When the developing thrombus causes complete occlusion and cessation of blood flow, an acute myocardial infarction ensues, heralded by ST-segment elevation on the electrocardiogram.

Coronary angiography utilizing contrast agents provides visualization of the heart and vasculature for patient diagnosis. Contrast media are currently used to produce images of organs and vessels with X-rays, magnetic resonance imaging and computed tomography. The current commercial X-ray contrast agents can be sorted into two categories: 1) ionic contrast agents, having an ionic carboxyl group and 2) non-ionic contrast agents, which do not contain any ionic groups. Examples of commercially available ionic contrast agents include Hypaque (Diatrizoate) and Hexabrix (Ioxaglate), while non-ionic agents include Omnipaque (Iohexol), Isovue (Iopamidol), Optiray (Ioversol), and Visipaque (Iodixanol).

It was noted in the mid 1980's that non-ionic contrast media had produced clots in syringes and catheter during angiographic procedures. This sparked research to determine the effect of contrast media on hemostasis. Nonionic contrast media shows a higher incidence of thrombotic events than the ionic contrast media as noted by the required FDA labeling:
“All nonionic, iodinated contrast media currently available inhibit blood coagulation, in vitro, less than ionic contrast media. Clotting has been reported when blood remains in contact with syringes containing nonionic contrast media. Serious, rarely fatal, thromboembolic events causing myocardial infarction and stroke have been reported during angiographic procedures with both ionic and nonionic contrast media. Therefore, meticulous intravascular administration technique is necessary to minimize thromboembolic events.”

In comparison to nonionic CM, ionic CM has greater patient discomfort, creating a higher tendency for nausea, headaches and chest pain. The perfect CM has not been achieved to date.

Examples of commercially available MR contrast agents include Omniscan (Gadodiamide), Optimark (Gadoversetamide), Magnevist (Gadopentetate dimeglumine) and Prohance (Gadoteridol). These agents are not reported to have adverse events in the coagulation area, however there are hemolytic concerns which are described in all of the package inserts. An anticoagulant MR contrast agent would be beneficial in the new emerging field of MR angiography. Here the vessels are already compromised with plaque burdens and an anticoagulant MR agent could be beneficial.

### C. SUMMARY OF THE INVENTION

One aspect of the invention provides an anticoagulant contrast agent comprising an organic scaffolding moiety, an organic anticoagulant moiety, and an imaging moiety, wherein said scaffolding moiety functionally links said anticoagulant moiety to said imaging moiety, and wherein if the anticoagulant moiety is: 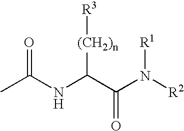
wherein n is two to six, R¹ and R² are independently hydrogen or alkyl or together form C4 to C8 alkylene, which is unsubstituted or substituted one to three times with lower alkyl or hydroxyl, and R³ is amino or guanidino, then the imaging moiety does not comprise iodine. Preferably, the anticoagulant inhibits an enzyme associated with blood clotting, e.g., thrombin, prothrombin, Factor Xa or Factor VIIa.

In a preferred embodiment, the anticoagulant moiety is a thrombin inhibitor comprising a P1 component, preferably an arginine or lysine mimetic. The thrombin inhibitor optionally comprises a P2 component and a P3 component. A preferred arginine mimetic has the structure: 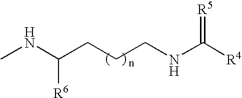
wherein, n is 0-3; R⁴ is SR₄ or NHR⁷, wherein (a) R is methyl or ethyl and (b) R⁷ is hydrogen, lower alkyl, halo, trihalomethyl, hydroxyl, alkoxy, amino, aryl, 5-membered heterocyclic ring, 2- or 3-ring fused heterocyclic system; R⁵ is O, S, or NR⁸, wherein R⁸ is hydrogen, lower alkyl, or hydroxyl; and R⁶ is formyl, alkylcarbonyl, amino, dialkoxyboronyl, dialkylphosphatidyl. More preferably, R⁷ is methyl, phenyl, fluoro, trifluoromethyl, propyl, hydroxyl, lower alkoxy NHCH₃, thiazole, or benzothiazole and R⁸ is hydrogen, methyl, ethyl or hydroxyl.

In yet another preferred embodiment, the anticoagulant contrast agent comprises an anticoagulant moiety that is a thrombin inhibitor comprising an arginine mimetic having the structure: 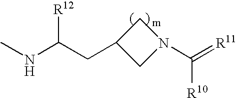
wherein m is 2 or 3; R¹⁰ is SR₄ or NHR¹³, wherein (a) R is methyl or ethyl and (b) R¹³ is hydrogen, lower alkyl, halo, trihalomethyl, hydroxyl, alkoxy, amino, a 5-membered heterocyclic ring or a 2- or 3-ring fused heterocyclic ring system; R¹¹ is O, S, or NR¹⁴, wherein R¹⁴ is hydrogen, lower alkyl, or hydroxyl; and R¹² is formyl, alkylcarbonyl, amino, dialkoxyboronyl, dialkylphosphatidyl.

In yet another preferred embodiment, the anticoagulant contrast agent comprises an anticoagulant moiety that is a thrombin inhibitor comprising an arginine mimetic having the structure: 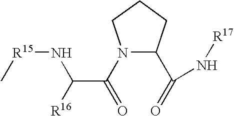
wherein, R¹⁵ is a covalent bond or SO₂; R¹⁶ is aryl; and R¹⁷ is a 5- or 6-membered heterocyclic radical substituted with CH₂NH₂ or has the structure: 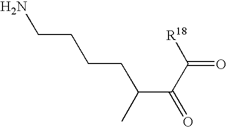
wherein R¹⁸ is amino or a 4- or 5-membered heterocyclic radical. Preferably R¹⁶ is phenyl.

In yet another preferred embodiment, the anticoagulant contrast agent will comprise an imaging moiety that comprises bromine or iodine. When the imaging agent comprises bromine or iodine, the scaffold, preferably, comprises one to three benzyl rings or a cage compound, e.g., a carborane cage, a fullerene, an adamantane or a diamantine. In one preferred embodiment the scaffold and imaging moiety together have the structure: 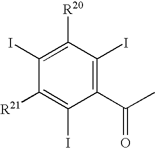
wherein, R²⁰ and R²¹ independently are amino or carbamoyl. In an alternative embodiment, the scaffold moiety and imaging moiety together have the structure: 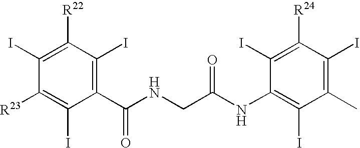
wherein, R²², R²³ and R²⁴ independently are amino, carbamoyl, or alkylcarbonyl.

Another preferred embodiment of the invention provides an anticoagulant contrast agent, wherein the imaging moiety comprises an electron-dense heavy metal, e.g., hafnium, tantalum, tungsten, rhenium, bismuth, or one of the lanthanide metals. When the imaging moiety is an electron-dense heavy metal, the scaffold moiety preferably is a metal chelator or a cage compound, e.g., a carborane cage, a fullerene, an adamantane, or a diamantine. Preferably the metal chelator is a derivative of H₃DO₃A-butrol.

A second aspect of the invention provides a method of visualizing an internal structure comprising (a) administering to a patient an amount of an anticoagulant contrast agent, as described above, and (b) exposing the internal structure to a diagnostic imaging procedure. If the diagnostic imaging procedure comprises an X-ray imaging procedure, preferably, the imaging moiety is iodine. If the diagnostic imaging procedure comprises an MRI imaging procedure, then the preferred imaging moiety is an electron-dense heavy metal or iodine. In a preferred embodiment, the visualized internal structure is a heart and the anticoagulant contrast agent is administered in conjunction with a catheterization procedure. In another preferred embodiment, the visualized internal structure is a kidney or liver and the anticoagulant contrast agent is administered intravenously to the patient. In yet another preferred embodiment, the visualized structure is the brain or other portion of the central nervous system and the anticoagulant contrast agent is administered intrathecally.

A third aspect of the invention provides an anticoagulant contrast media comprising an anticoagulant contrast agent, as described above, and a pharmaceutically acceptable carrier, preferably isotonic saline. The anticoagulant contrast media, optionally, further comprises an antiemetic agent, a tranquilizer or a muscle relaxant.

### III. BRIEF DESCRIPTION OF THE FIGURES

Exemplary Factor Xa inhibitors are illustrated in FIG. 1.

FIG. 2 illustrates a few examples of FVIIa inhibitors.

FIG. 3 illustrates the D-Phe-Pro-Arg motif that mimics the natural substrate of thrombin.

FIG. 4 provides a number of known arginine mimetics.

FIG. 5 illustrates non-basic moieties that may be used as a P1 component of a thrombin inhibitor.

FIG. 6 provides preferred P2 components of a thrombin inhibitor that may be used as an anticoagulant moiety in the present invention.

FIG. 7 illustrates preferred P3 component of a thrombin inhibitor that may be used as an anticoagulant moiety in the present invention.

FIG. 8A-8E provides several exemplary anticoagulant contrast agents that contain at least 35% iodine (by weight).

FIG. 9A-B illustrates iodinated icosahedral boron cage compounds, which can contain a minimum of six iodines (preferably 8-11 iodine atoms) and have greater than 65% iodine by weight (up to 90%). For simplicity, only the first figure exemplifies the imaging element (shaded squares). Carbons are illustrated by black circles; borane is illustrated by a white circle.

FIG. 10 illustrates preferred polar groups used to enhance the solubility of endohedral fullerene compounds as well as several possible attachment sites when an anticoagulant is attached to the fullerene scaffold. In these illustrations and at other locations in this text, fullerene itself is represented by “C₆₀” appearing in a circle.

FIG. 11 provides some common metal chelators that can be used in conjunction with the present invention.

### IV. DETAILED DESCRIPTION

A. Overview

The present invention provides novel anticoagulant contrast agents, which comprise an organic scaffolding moiety, an organic anticoagulant moiety, and an imaging moiety. The invention also provides anticoagulant contrast media and methods of visualizing internal structures utilizing the novel anticoagulant contrast agents and anticoagulant contrast media.

B. Definitions

The term “alkoxy” refers to a monovalent radical of the formula RO—, where R is an alkyl as defined herein. Lower alkoxy refers to an alkoxy of 1-6 carbon atoms, with higher alkoxy is an alkoxy of seven or more carbon atoms. Representative lower alkoxy radicals include, but are not limited to, methoxy, ethoxy, n-propoxy, n-butoxy, n-pentyloxy, n-hexyloxy, isopropoxy, isobutoxy, isopentyloxy, amyloxy, sec-butoxy, tert-butoxy, tert-pentyloxy. Higher alkoxy radicals include those corresponding to the higher alkyl radicals set forth herein. The radical optionally is substituted with substituents at positions that do not significantly interfere with the preparation of compounds falling within the scope of this invention and that do not significantly reduce the efficacy of the compounds.

The term “alkyl” refers to a monovalent, saturated aliphatic hydrocarbon radical having, in cases where so specified, the indicated number of carbon atoms. For example, a “C 1-6 alkyl” or an “alkyl of 1-6 carbons” or “Alk 1-6” would refer to any alkyl group containing one to six carbons in the structure. “C 1-20 alkyl” refers to any alkyl group having one to twenty carbons. Alkyl may be a straight chain (i.e. linear) or a branched chain. Lower alkyl refers to an alkyl of 1-6 carbons. Representative examples include lower alkyl radicals include methyl, ethyl, n-propyl, n-butyl, n-pentyl, n-hexyl, isopropyl, isobutyl, isopentyl, amyl, sec-butyl, tert-butyl, and tert-pentyl. Higher alkyl refers to alkyls of seven carbons and above. These include n-heptyl, n-octyl, n-nonyl, n-decyl, n-dodecyl, n-tetradecyl, n-hexadecyl, n-octadecyl, and n-eicosyl, along with branched variations thereof. The radical optionally is substituted with substituents at positions that do not significantly interfere with the preparation of compounds falling within the scope of this invention and that do not significantly reduce the efficacy of the compounds. The alkyl optionally is substituted with one to five substituents independently selected from the group consisting of halo, lower alkoxy, hydroxy, cyano, nitro, or amino.

The term “alkylcarbonyl” is a monovalent radical having the formula —C(O)Alk, where Alk is alkyl, preferably lower alkyl.

“Alkylene” by itself or as part of another substituent refers to a saturated straight-chain alkyl di-radical derived by the removal of a hydrogen atom from each of the terminal carbon atoms of a parent alkane. If each radical is attached to the same atom, a cyclic structure is formed. A “C4 to C8 alkylene” or an “alkylene of 4-8 carbons” would refer to any alkylene group containing four to eight carbons in the structure.

“Amino” by itself or as part of another substituent refers to the radical —NRR′ where R and R′ independently are hydrogen, alkyl, cycloalkyl or aryl as defined herein.

The term “anticoagulant moiety” refers to a chemical moiety that hinders the clotting of blood. In some cases the anticoagulant moiety is a radical obtained from a known anticoagulant by removing a hydrogen atom, preferably from a carbon, and the anticoagulant contrast media is formed by attaching said contrast moiety to a scaffolding moiety by a bond at the position of the removed hydrogen. In some cases, the entire anticoagulant contrast agent molecule will function as an anticoagulant, wherein removal of any portion of the molecule will interfere with the anticoagulant function of the contrast agent.

The term “arginine mimetic” refers to a chemical moiety that bears a physical or functional resemblance to arginine, such that it can replace arginine without complete loss of function or disruption of a naturally occurring biochemical interaction.

“Aryl” by itself or as part of another substituent refers to a monovalent aromatic hydrocarbon radical derived by the removal of one hydrogen atom from a single carbon atom of a parent aromatic ring system. Typical aryl groups include, but are not limited to, phenyl, 1-naphthyl, 2-naphthyl. A “1-naphthyl” or “2-naphthyl” is a radical formed by removal of a hydrogen from the 1- or 2-position of a naphthalene structure, respectively. It optionally is substituted with from one to four substituents independently selected from the group consisting of halo, lower alkyl, lower alkoxy, hydroxy, cyano, nitro, amino, halogenated lower alkyl, formyl, halogenated lower alkoxy, hydroxycarbonyl, lower alkoxycarbonyl, lower alkylcarbonyloxy, and lower alkylcarbonylamino. A “phenyl” is a radical formed by removal of a hydrogen from a benzene ring. The phenyl optionally is substituted with from one to five substituents independently selected from the group consisting of halo, lower alkyl, lower alkoxy, hydroxy, cyano, nitro, amino, halogenated lower alkyl, halogenated lower alkoxy, carbonyl, hydroxycarbonyl, lower alkylcarbonyloxy, benzyloxy, optionally substituted piperidino, lower alkoxycarbonyl, and lower alkylcarbonylamino.

The term “contrast agent” refers to a compound that increases the degree of difference between the lightest and darkest parts of medical scan, preferably an X-ray or MRI, relative to a medical scan performed without the use of a contrast agent.

The term “contrast media” refers to a formulation, suitable for administration to a patient, comprising a contrast agent and a carrier and/or excipient. The term “media” is used to indicate either the singular or plural form, depending on the context of the sentence in which it is used, or can be used to indicate both.

The term “cycloalkyl” refers to a monovalent, alicyclic, saturated hydrocarbon radical having three or more carbons forming the ring. While known cycloalkyl compounds may have up to 30 or more carbon atoms, generally there will be three to seven carbons in the ring. The latter include, for example, cyclopropyl, cyclobutyl, cyclopentyl, cyclohexyl, and cycloheptyl. The radical optionally is substituted with substituents at positions that do not significantly interfere with the preparation of compounds falling within the scope of this invention and that do not significantly reduce the efficacy of the compounds. The cycloalkyl optionally is substituted with one to five substituents independently selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino.

The term “dialkyloxyboronyl” by itself or as part of another substituent refers to the radical —B(OR)₂ wherein each R independently is hydrogen or lower alkyl.

The term “dialkylphosphatidyl” by itself or as part of another substituent refers to the radical —PO(OR)₂ wherein each R independently is hydrogen or lower alkyl.

The term “guanidino” by itself or as part of another substituent refers to the radical —NH—C(:NH)NH₂—.

The term “imaging moiety” refers to (1) an electron-dense atom that absorbs X-rays or (2) a paramagnetic heavy metal ion that alters the spin relaxation time of a tissue or internal structure. The imagining moiety used for MR contrast agents can have one of two effects on signal intensity: (1) positive enhancement of longitudinal relaxation; or (2) negative enhancement of transverse relaxation. A contrast agent of the present invention will contain at least one imaging moiety, preferable 1 to 12, imaging moieties per molecule. Paramagnetic materials include oxygen and ions of various metals like Fe, Mg, and Gd. These ions have unpaired electrons, resulting in a positive magnetic susceptibility. The effect on MRI is increase in the T1 and T2 relaxation rates (decrease in the T1 and T2 times).

The term “lysine mimetic” refers to a chemical moiety that bears a physical or functional resemblance to lysine, such that it can replace lysine without complete loss of function or disruption of a naturally occurring biochemical interaction.

A “4-membered heterocyclic ring” is a monovalent radical of a 4-member closed ring containing carbon and at least one other element, generally nitrogen, oxygen, or sulfur and may be fully saturated, partially saturated, or unsaturated. Generally the heterocycle will contain no more than two hetero atoms. Representative examples of 4-membered heterocycles include cyclobutanyl, cyclobutadienyl, azetidinyl, 1,2-dihydro-azetyl, oxetanyl, thietanyl and thietyl. The corresponding fully saturated and partially saturated radicals are also included. The radical optionally is substituted with substituents at positions that do not significantly interfere with the preparation of compounds falling within the scope of this invention and that do not significantly reduce the efficacy of the compounds. The ring optionally is substituted with one substituent selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino.

A “5-membered heterocyclic ring” is a monovalent radical of a 5-member closed ring containing carbon and at least one other element, generally nitrogen, oxygen, or sulfur and may be fully saturated, partially saturated, or unsaturated. Generally the heterocycle will contain no more than two hetero atoms. Representative examples of unsaturated 5-membered heterocycles with only one hetero atom include 2- or 3-pyrrolyl, 2- or 3-furanyl, and 2- or 3-thiophenyl. Corresponding partially saturated or fully saturated radicals include 3-pyrrolin-2-yl, 2- or 3-pyrrolidinyl, 2- or 3-tetrahydrofuranyl, and 2- or 3-tetrahydrothiophenyl. Representative unsaturated 5-membered heterocyclic radicals having two hetero atoms include, but are not limited to, imidazolyl, oxazolyl, thiazolyl, pyrazolyl, tetrazolyl. The corresponding fully saturated and partially saturated radicals are also included. The radical optionally is substituted with substituents at positions that do not significantly interfere with the preparation of compounds falling within the scope of this invention and that do not significantly reduce the efficacy of the compounds. The ring optionally is substituted with one or two substituents selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino.

A “6-membered heterocyclic ring” is a monovalent radical of a 6-member closed ring containing carbon and at least one other element, generally nitrogen, oxygen, or sulfur and may be fully saturated, partially saturated, or unsaturated. Generally the heterocycle will contain no more than two hetero atoms. Representative examples of unsaturated 6-membered heterocycles with only one hetero atom include 2-, 3-, or 4-pyridinyl, 2H-pyranyl, and 4H-pryanyl. Corresponding partially saturated or fully saturated radicals include, but are not limited to, 2-, 3-, or 4-piperidinyl, 2-, 3-, or 4-tetrahydropyranyl. Representative unsaturated 6-membered heterocyclic radicals having two hetero atoms include, but are not limited to, 3- or 4-pyridazinyl, 2-, 4-, or 5-pyrimidinyl, 2-pyrazinyl. The corresponding fully saturated and partially saturated radicals are also included, e.g. 2-piperazine. The radical optionally is substituted with substituents at positions that do not significantly interfere with the preparation of compounds falling within the scope of this invention and that do not significantly reduce the efficacy of the compounds. The ring optionally is substituted with one or two substituents selected from the group consisting of halo, lower alkyl, lower alkoxy, hydroxy, cyano, nitro, amino, halogenated lower alkyl, halogenated lower alkoxy, hydroxycarbonyl, lower alkoxycarbonyl, lower alkylcarbonyloxy, and lower alkylcarbonylamino.

“Visualization-effective amount” means the amount of a compound that, when administered to a patient, is sufficient to visualize the desired internal organ or structure. The “visualization-effective amount” will vary depending on the compound, the organ/structure and its size and the age, weight, etc., of the patient to be scanned.

C. Anticoagulant Contrast Agents

Anticoagulant contrast agents combine two technologies, anticoagulants and contrast agents, into a single active compound. Thus, anticoagulant contrast agents will perform two clinical functions, visualization of an internal structure or organ and prevention of clot formation within the blood vessels. The anticoagulant contrast agents of the invention are composed of at least three components: 1) an anticoagulant moiety, 2) an imaging moiety and 3) a supporting organic scaffold moiety.

The anticoagulant contrast agent, preferably, will directly or indirectly inhibit thrombin, thereby inhibiting both primary hemostasis (platelet activation, degranulation, adhesion and aggregation) and soluble phase coagulation (fibrin formation). Thrombin is the final protease in the coagulation cascade, thus if you inhibit the process from above, i.e. by inhibiting FXa or FVIIa it will alter the production of thrombin hence its activities. Arterial thrombi are primarily composed of platelets and fibrin; thus the local injection of a thrombin inhibitor could significantly reduce thrombotic complications. Current anticoagulant therapies are administered systemically and may not be preventative at the site where the imaging agent is injected into the vasculature. Additionally, heparin and low molecular weight heparin (LMWH) cannot inhibit fibrin bound thrombin. This bound thrombin is still functional and can activate proteins and platelets.

An exemplary anticoagulant contrast agent would comprise a thrombin inhibitor (anticoagulant moiety), iodine (imaging moiety), and benzene (supporting organic scaffold moiety).

1. Anticoagulant Moieties

A variety of anticoagulant derivatives can be attached to the scaffold and imaging moieties. A wide variety of diagnostic imaging moieties and scaffolds are known in the art, e.g., as embodied in commercial contrast agents (Diatrizoate, Metrizoate or Iothalamate). See also, Remington's Pharmaceutical Sciences, 18^{th} Ed., Chapter 65: Diagnostic Drugs, © 1990.

There has been an intense effort to discover small molecule anticoagulants that work to specifically inhibit serine proteases in the blood coagulation cascade. Direct inhibition of thrombin has been well studied and has resulted in promising clinical drug candidates. Other important targets of anticoagulants are Factor Xa, Factor VIIa, Factor IXa and Factor XIa.

Small molecule inhibitors can be designed based on coupling P1-P2-P3 residues that “fit” within the binding pockets of the desired serine protease. For example, if a thrombin inhibitor is being designed, the P1 residue is modeled to interact with an aspartic acid in the S1 pocket. Thus a basic residue such as guanidinium or amine is typically used. Regardless of the serine protease targeted, the small molecule serine protease inhibitor is coupled to an imaging moiety/scaffold to create an anticoagulant contrast agent.

In one preferred embodiment, the anticoagulant moiety is a Factor Xa inhibitor. Factor Xa complexes with Factor Va and calcium ions on a phospholipid surface to activate prothrombin to thrombin. This complex is referred to as the prothrombinase complex which is the convergence of the intrinsic and extrinsic blood coagulation pathways. Factor Xa inhibitors exert their anticoagulant effect by directly slowing the generation of thrombin. Exemplary Factor Xa inhibitors are illustrated in FIG. 1. The anticoagulant, a Factor Xa inhibitor, can be attached to the scaffold moiety at the P1, P2, P3 or P4 residues.

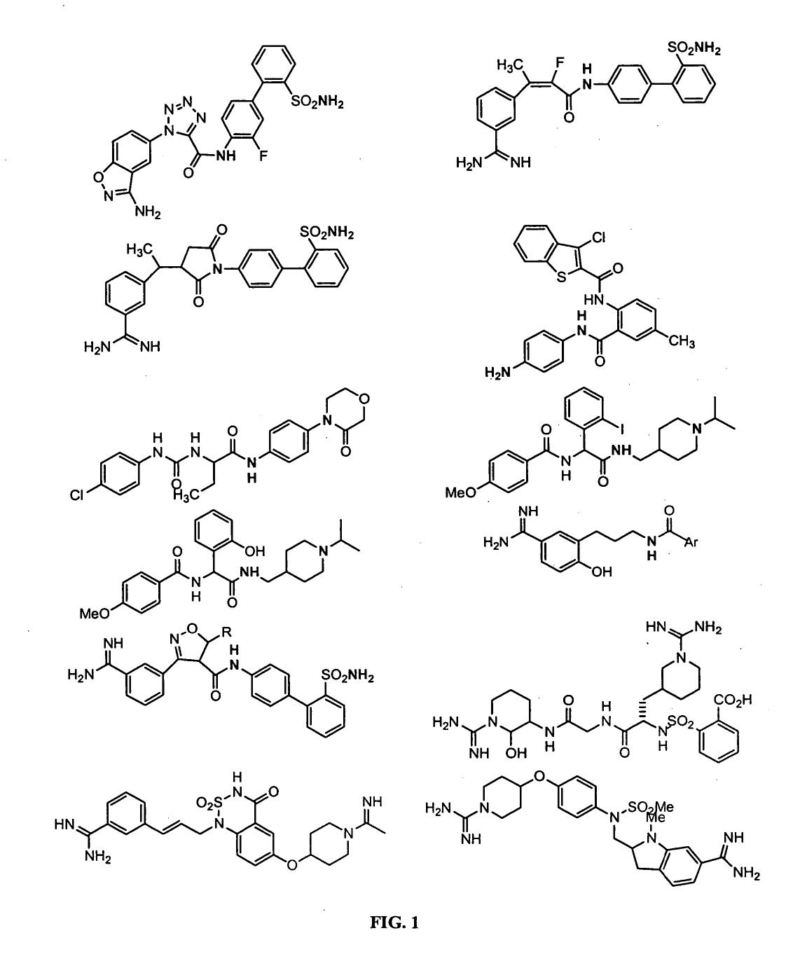

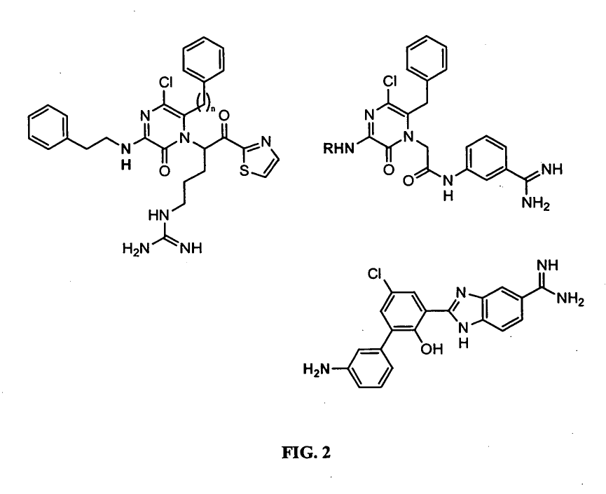

In another preferred embodiment, the anticoagulant moiety inhibits Factor VIIa. Attention has been drawn recently to Factor VIIa because of its role as initiator of the cascade in complex with tissue factor (TF) upon blood vessel damage. FIG. 2 illustrates a few examples of FVIIa inhibitors. Coupling can occur at P1, P2 or P3 residues.

In yet another preferred embodiment, the anticoagulant moiety inhibits thrombin. Thrombin is a multifunctional serine protease with trypsin-like specificity, which plays a central role in hemostasis by regulating the blood coagulation cascade and platelet activation. Serving as the terminal enzyme of the coagulation pathway, thrombin cleaves fibrinogen to fibrin, which in combination with Factor XIIIa and platelets aggregates to a gel-like matrix, ultimately leading to the formation of blood clots.

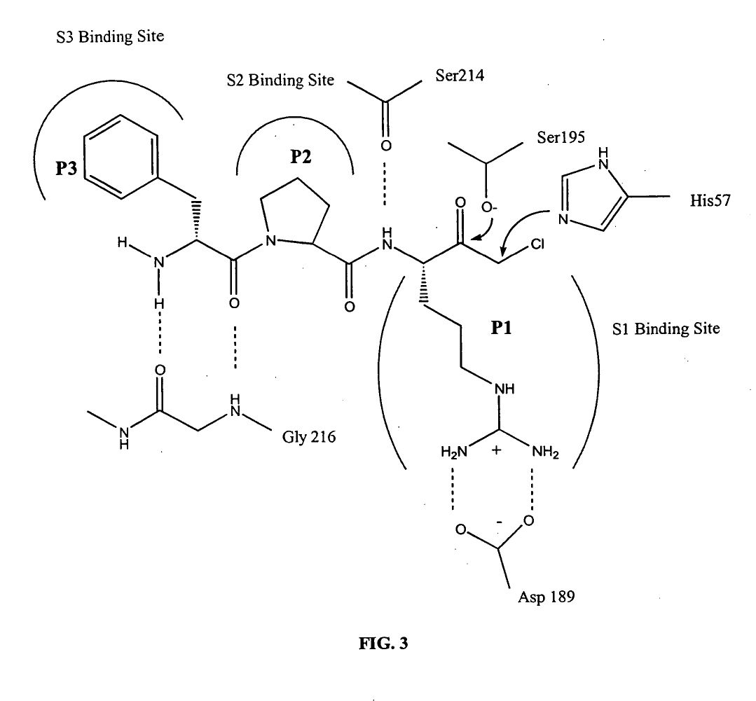

A contrast agent that inhibits thrombin can be achieved by incorporating the thrombin inhibiting moiety into the imaging agent. Many approaches to thrombin inhibitors are based on the D-Phe-Pro-Arg motif that mimics the natural substrate shown in FIG. 3. Thrombin contains three sites, S1, S2 and S3, that bind with moieties within the substrate, P1, P2 and P3. Specific, hydrophobic, noncovalent interactions occur in binding sites S2 and S3. The enzyme S1 site of thrombin is a deep pocket with Asp 189 at its bottom, capable of forming ionic and hydrogen interactions with positively charge residues such as arginine and lysine. The role of each substrate component, P1, P2 and P3 has been extensively examined and a variety of structures can be accommodated in sites S1, S2 and S3. Selectivity for thrombin inhibition can be achieved by maximizing interactions within these binding sites and incorporating groups that do not fit within other similar serine-protease binding sites. A contrast agent that inhibits thrombin can be achieved by attaching the thrombin inhibiting moiety (P1, P2-P1 or P3-P2-P1) into the scaffold moiety.

a. Thrombin Inhibitors

The anticoagulant component of the anticoagulant contrast agent can be rationally designed based on existing thrombin literature and attached to the scaffold moiety. The search for potent thrombin inhibitors for antithrombotic therapy has received much attention in the field of medicinal chemistry. In the past, synthetic efforts have been concentrated on tripeptide analogues and arginine amide derivatives. These are often referred to as peptide-mimetics. In recent years, new types of non-peptidic thrombin inhibitors have been described having heterocyclic core structures.

Peptidomimetics have found wide application as bioavailable, biostable and potent mimetics of naturally occurring biologically active peptides. L-Arginine is a guanidino group-containing a basic amino acid which is positively charged at neutral pH and is involved in many important physiological and pathological processes. Many enzymes display a preference for the arginine residue that is found in many natural substrates and in synthetic inhibitors of many trypsin-like serine proteases such as thrombin, Factor Xa and trypsin. The highly basic guanidino moiety incorporated in enzyme inhibitors is often associated with low selectivity. A significant effort has focused on arginine mimetics with reduced basicity that confers selective inhibition. A thorough review of arginine mimetics was written by Peterlin-Masic and Kikelj (“Arginine Mimetics,” Tetrahedron 57:7073-7105 (2001)).

One group of preferred arginine mimetics comprise a modified guanidino moiety. Exemplary arginine mimetics comprise the structure: 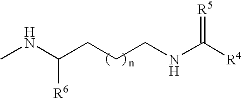
wherein
n is 0-3;
R⁴ is SR₄ or NHR⁷, wherein R is methyl or ethyl, and R⁷ is hydrogen, lower alkyl, halo, trihalomethyl, hydroxyl, alkoxy, amino, aryl, 5-membered heterocyclic ring, or 2- or 3-ring fused heterocyclic system;
R⁵ is O, S, or NR⁸, wherein R⁸ is hydrogen, lower alkyl, or hydroxyl; and
R⁶ is formyl, alkylcarbonyl, amino, dialkoxyboronyl, dialkylphosphatidyl.
   More preferably R⁷ is methyl, phenyl, F, CF₃, propyl, hydroxyl, lower alkoxy, NHCH₃, thiazole, or benzothiazole and R⁸ is hydrogen, methyl, ethyl or hydroxyl.

Another preferred arginine mimetic useful in the present invention has the structure: 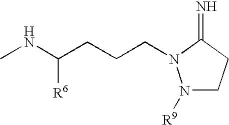
wherein, R⁹ is hydrogen or lower alkyl and R⁶ is formyl, alkylcarbonyl, amino, dialkoxyboronyl, dialkylphosphatidyl.

Another preferred arginine mimetic comprises a conformationally constrained modified guanidino moiety having the structure: 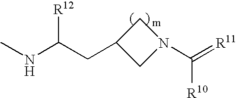
wherein
m is 2 or 3;
R¹⁰ is SR₄ or NHR¹³, wherein R is methyl or ethyl and R¹³ is hydrogen, lower alkyl, halo, trihalomethyl, hydroxyl, alkoxy, amino, a 5-membered heterocyclic ring or a 2- or 3-ring fused heterocyclic ring system;
R¹¹ is O, S, or NR¹⁴, wherein R¹⁴ is hydrogen, lower alkyl, or hydroxyl; and
R¹² is formyl, alkylcarbonyl, amino, dialkoxyboronyl, dialkylphosphatidyl.

Yet another preferred arginine mimetic comprises a conformationally constrained modified guanidino moiety having the structure: 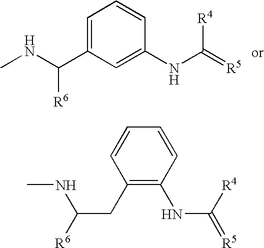
wherein
R⁶ is formyl, alkylcarbonyl, amino, dialkoxyboronyl, dialkylphosphatidyl;
R⁴ is SR₄ or NHR⁷, wherein R is methyl or ethyl, and R⁷ is hydrogen, lower alkyl, halo, trihalomethyl, hydroxyl, alkoxy, amino, aryl, 5-membered heterocyclic ring, 2- or 3-ring fused heterocyclic system; and
R⁵ is O, S, or NR⁸, wherein R⁸ is hydrogen, lower alkyl, or hydroxyl.

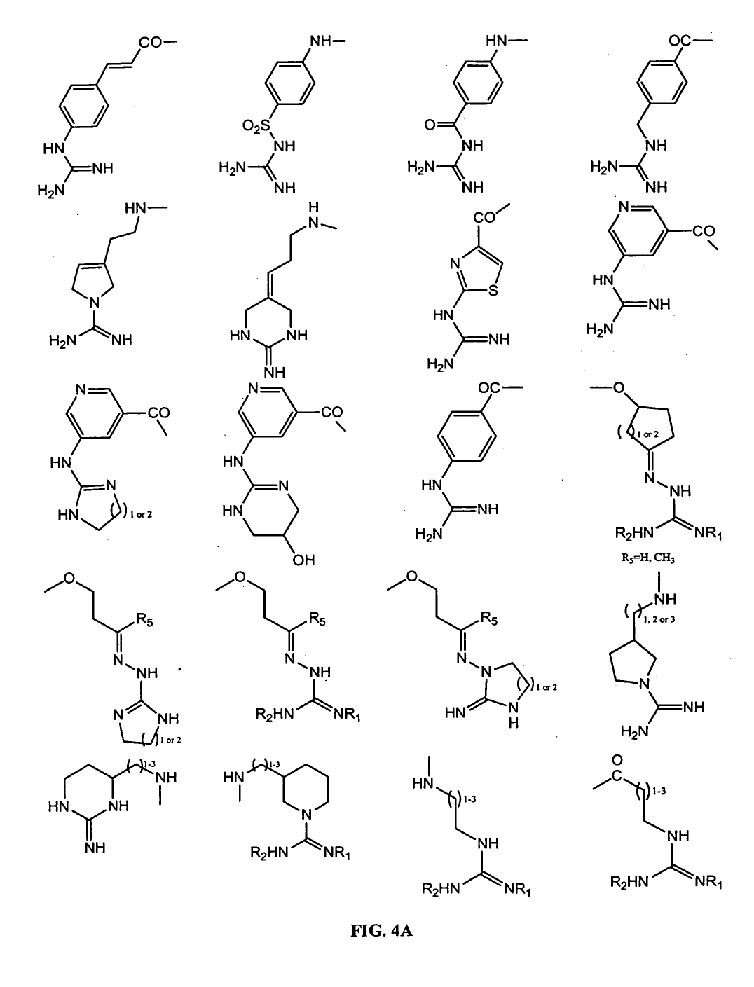

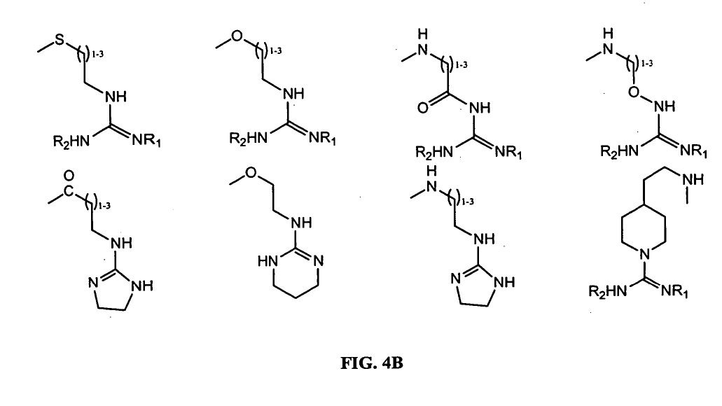

The anticoagulant used in the anticoagulant contrast media of the invention may also comprise any one of the know arginine mimetics illustrated in FIG. 4.

While arginine has often been the target amino acid for the design of a thrombin inhibitor, thrombin binds to protein substrates at both arginine and lysine residues. Thus, lysine derivatives and mimetics can also be used to develop thrombin inhibitors. In a preferred embodiment, the anticoagulant moiety will comprise a lysine mimetic having the structure: 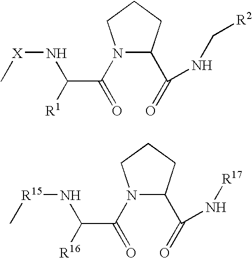
wherein, R¹⁵ is a covalent bond or SO₂; R¹⁶ is aryl; and R¹⁷ is a 5- or 6-membered heterocyclic radical substituted with CH₂NH₂ or has the structure: 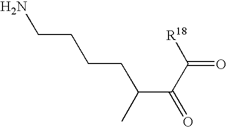
wherein R¹⁸ is amino or a 4- or 5-membered heterocyclic radical. Preferably R¹⁶ is phenyl.

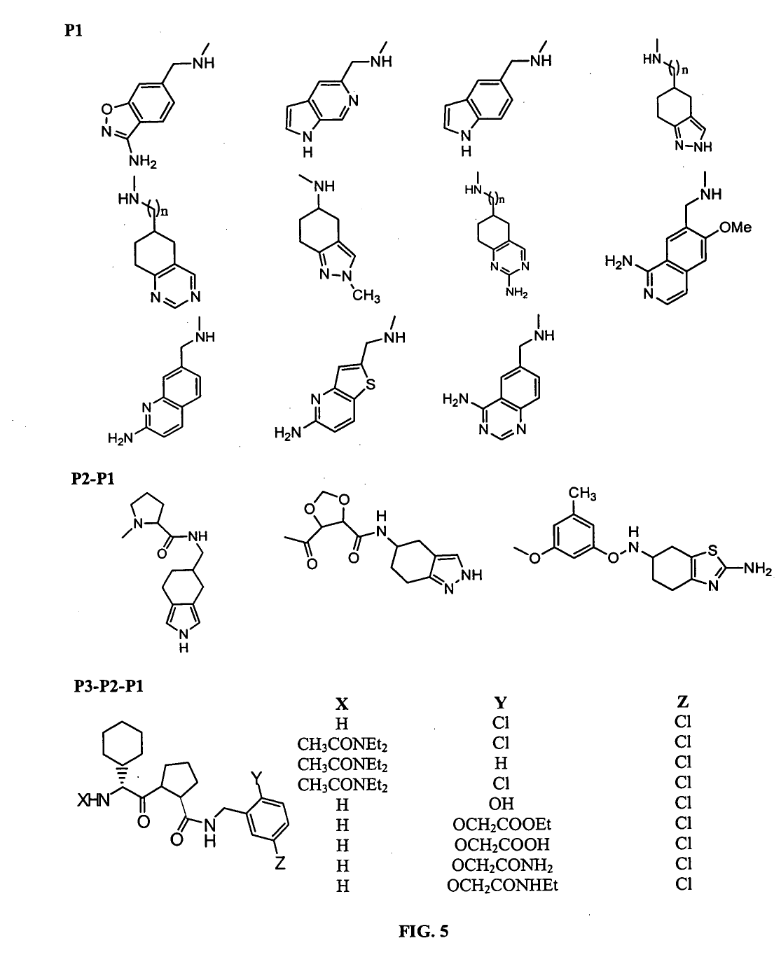

Non-basic P1 components, such as those illustrated in FIG. 5 also can be used in designing an anticoagulant moiety that inhibits thrombin.

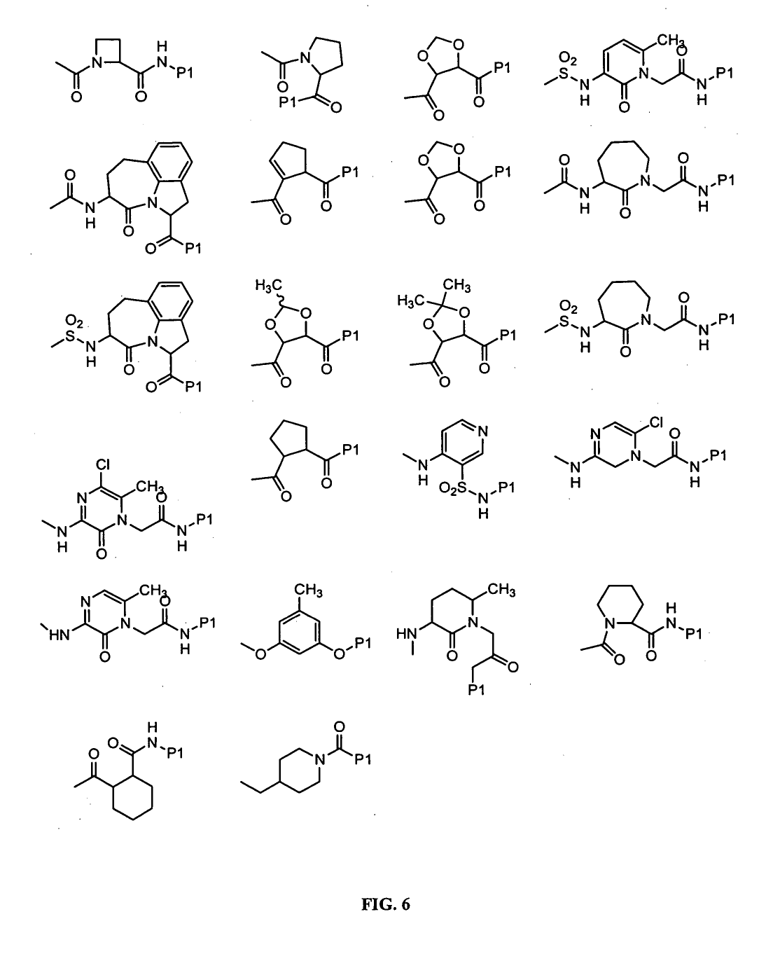

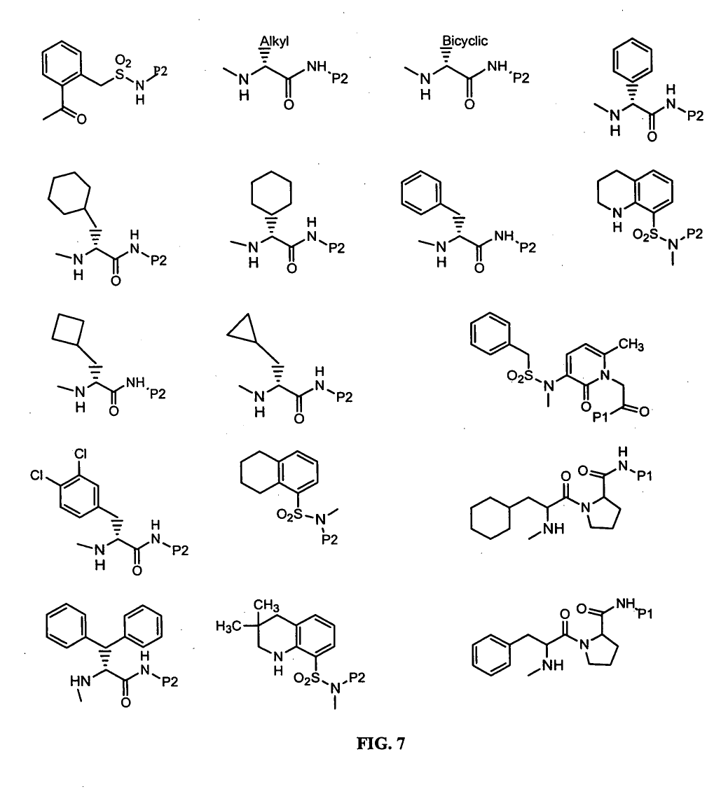

The thrombin inhibitor used in construction of an anticoagulant contrast agent can be a P1, P1-P2, or P1-P2-P3 thrombin inhibitor. A P2 component will bind to the S2 site of thrombin. Particularly preferred P2 components for use in the present invention are illustrated in FIG. 6. A P3 component will bind to the S3 site of thrombin. Preferred P3 components for use in the present invention are illustrated in FIG. 7.

2. Imaging and Scaffold Moieties

X-ray contrast media has been developed over the years to improve the following features a) visualization b) solubility, c) viscosity, d) osmolality and e) toxicity. Exemplary contrast agents are grouped according to chemical structure (Table 1).

| Osmolality | High | Low | | Iso |
|---|---|---|---|---|
| (mOsm/kg) | (>1500) | (600-1000) | | (280) |
| Ionicity | Ionic | Ionic | Nonionic | Nonionic |
| Benzene Rings | Monomer | Dimer | Monomer | Dimer |
| Commercial | Diatrizoate | Ioxaglate | Iohexol | Iodixanol |
| Products | | | Iopamidol | |
| | | | Ioversol | |
| | | | Iopromide | |
| | | | Iopentol | |
| | | | Iomerpol | |
| Viscosity (cps) | 14 | 15 | 10-20 | 26 |
| Adverse effects | Nausea | Nausea | Thrombotic | Thrombotic |
| | Heat | Heat | Potential | Potential |
| | Sensation | Sensation | | |
| | Chest Pain | Chest Pain | | |
| | Headache | Headache | | |

Ionic contrast agents contain a free carboxyl group whereas nonionic contrast agents do not contain a charged group. The groups are defined further with dimeric contrast agents containing two iodinated benzene rings and monomeric contrast agents containing one iodinated benzene ring.

Approximately 30-40% of the more than 7-10 million MR examinations performed in the world each year are accompanied by administration of a contrast agent. The gadolinium chelates constitute the largest group of MR contrast media and are considered to be very safe. As with X-ray imaging agents, there are two groups of imaging agents, ionic and nonionic. These agents are also classified according to the structure of the chelating compound, either linear or macrocycle. These gadolinium MR imaging agents have similar effectiveness and safety profiles. Some exemplary commercially available MR contrast agents are described in Table 2.

| Trade | Proprietary | Chelator | | Osmolality | Viscosity |
|---|---|---|---|---|---|
| Name | Name | Structure | Ionicity | (mmol/kg) | 37° C. (mPa) |
| Magnevist | Gd-DTPA | Linear | Ionic | 1960 | 2.9 |
| Gadovist | Gadobutrol | Macrocylce | Non-ionic | 1603 | 4.9 |
| Dotarem | Gd-DOTA | Macrocycle | Ionic | 1350 | 2.0 |
| Omniscan | Gd-BMA | Linear | Non-ionic | 789 | 1.4 |
| Prohance | Gd-DO3A | Macrocycle | Non-ionic | 630 | 1.3 |
| MulitHance | Gd-BOPTA | Linear | Ionic | 1970 | 5.3 |
| OptiMark | Gd-DTPA-BMEA | Linear | Nonionic | 1110 | 2.0 |

a. Halogen

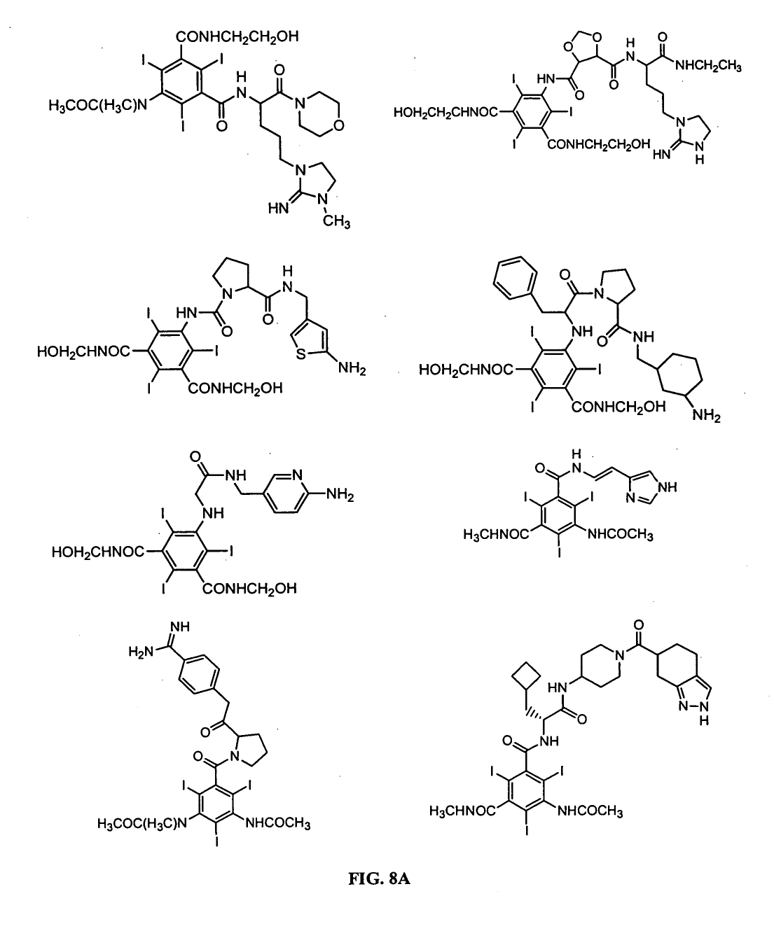

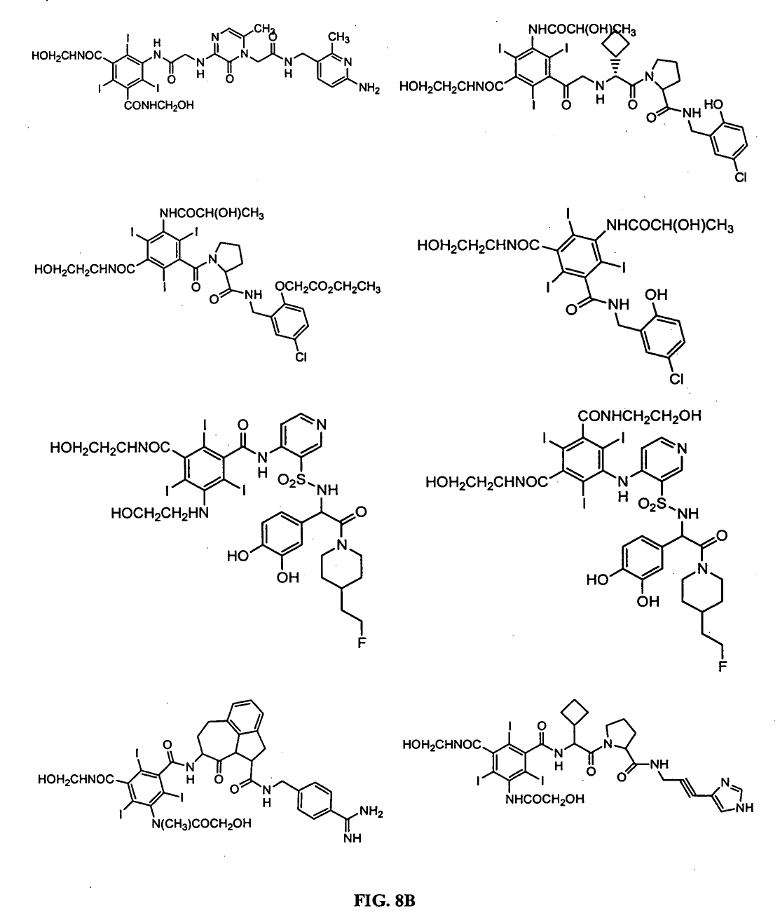

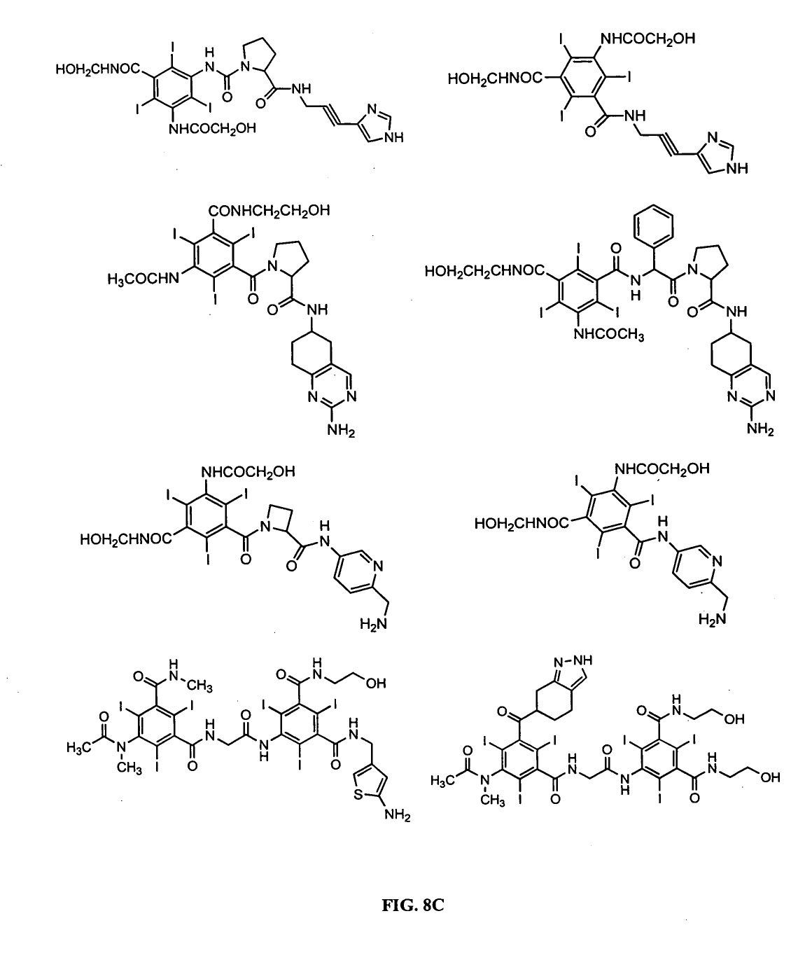

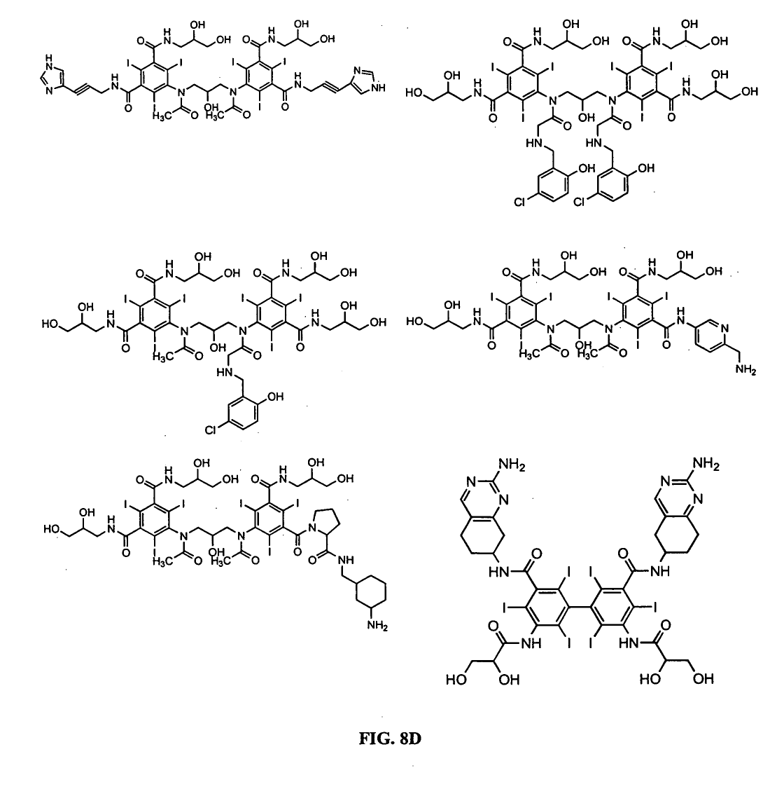

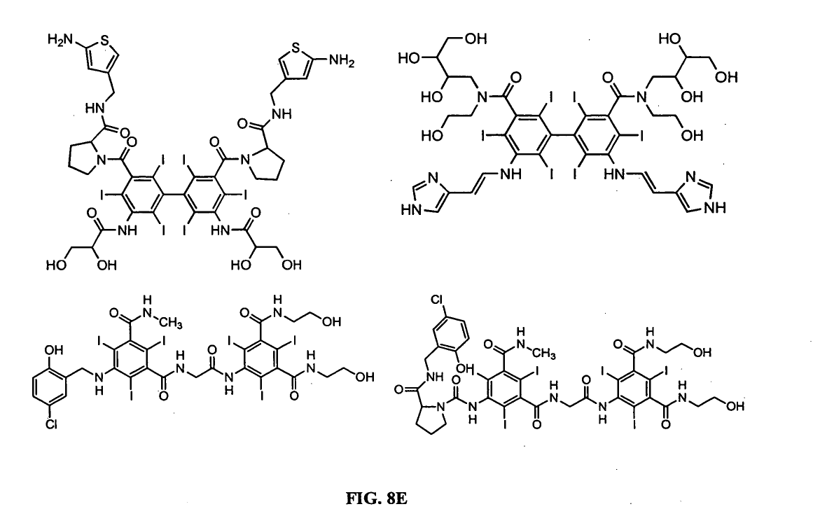

The most typical imaging moieties comprise halogen, particularly bromine or iodine. Especially common are contrast agents comprising an iodinated benzene component containing one, two, or three tri-iodinated benzene rings. As used in the present invention, iodine is the imaging moiety and the benzene ring is the scaffold moiety. Iodinated benzyl rings can be prepared using methods known in the chemical arts. For example, benzyl rings can be converted to the corresponding iodo-compound by iodination with a suitable agent, such as iodine monochloride, with I₂ in the presence of K₁ and C₂H₅NH₂, with I₂ dissolved in oleum, with an I₂/H₅IO₆ mixture or potassium iododichloride (KICl₂). The side chain groups on the iodinated benzene ring preferably are substituted to maximize solubility. The resulting anticoagulant contrast agent preferably contains at least 35% iodine (by weight) for good imaging capabilities. Exemplary anticoagulant contrast agents possessing these properties are shown in FIG. 8A-D.

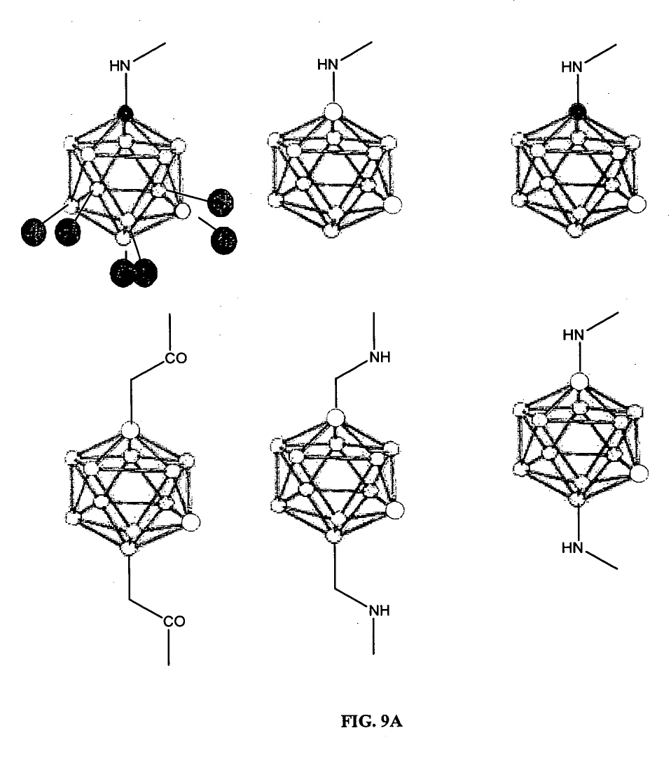

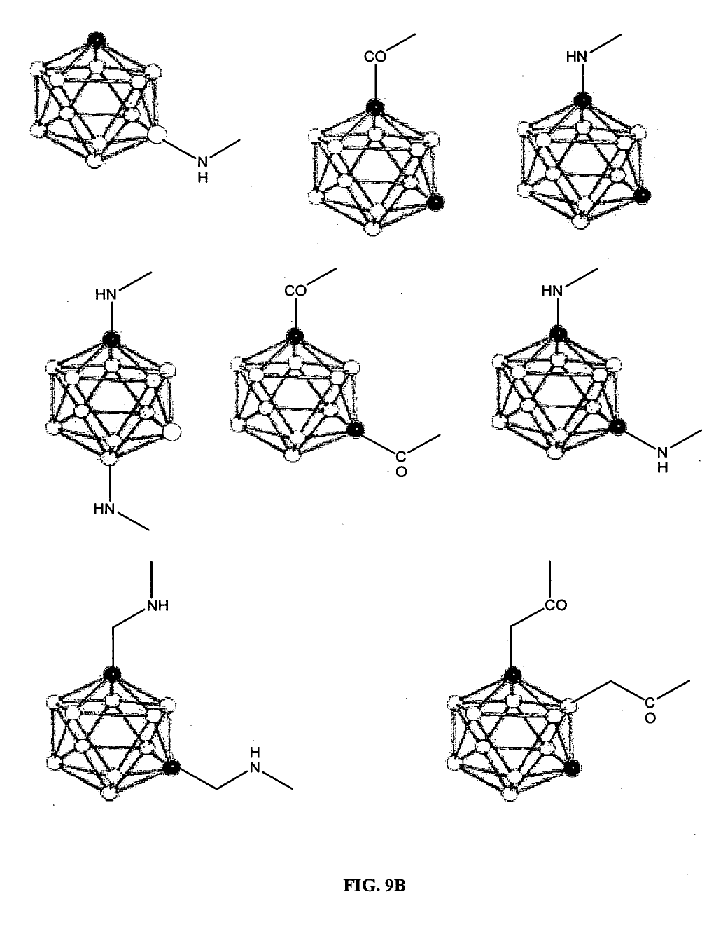

One method of improving contrast agents is to increase the iodine content in the molecules. Iodinated borane and carborane cage compounds (illustrated in FIG. 9A-B) can contain a minimum of six iodines (preferably 8-11 iodine atoms) and have greater than 65% iodine by weight (up to 90% possible) in comparison to commercial iodinated benzene rings compounds that contain 28-50% iodine by weight. Boron-iodine bonds are stronger than carbon-iodine bonds. Iodinated borane and carborane cage molecules are particularly stable to chemical and biological deiodination. Borane and carborane cages exist as anionic, cationic or neutral compounds. Srivastava, et al. Synthesis of Highly Iodinated Icosahedral Mono- and Dicarbon Carboranes, J. Org. Chem. 61:9041-9044 (1996). See also U.S. Pat. No. 5,679,322.

Another preferred scaffold moiety includes cage compounds (i.e., a molecule in which a plurality of rings formed by covalently bound atoms define a volume, such that an ion located within the volume can not leave the volume by passing through a ring), and especially contemplated cage compounds are adamantane, diamantane and fullerenes. Nakamura, Sawamura, and coworkers reported the preparation of pentahaptofullerene metal complexes (see, e.g., J. Am. Chem. Soc. 118 (1996) 12850 and Chem. Letters (2000) 270). Conjugate addition using a large excess (25-60 equivalents) of an organocuprate reagent, followed by inverse quenching, gives a pentaalkylated cyclopentadienyl precursor. The precursor can be deprotonated and combined with a transition metal source to generate a fullerene-cyclopentadienyl complex. Preferably, a fullerene is coupled to a moiety comprising an iodinated benzyl ring, e.g., iohexol. More preferably, the fullerene is coupled to iohexol, and an additional functional group, which enhances solubility of the molecule as diagrammed below:

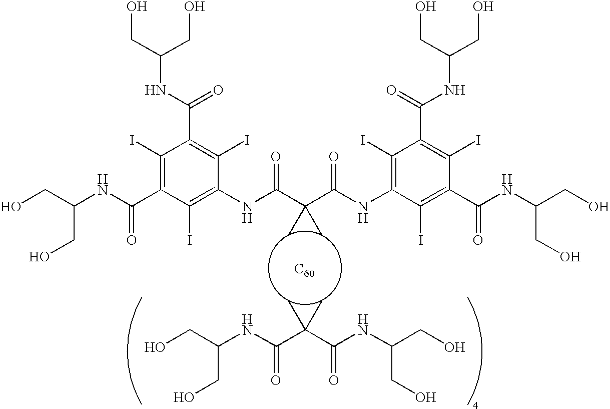

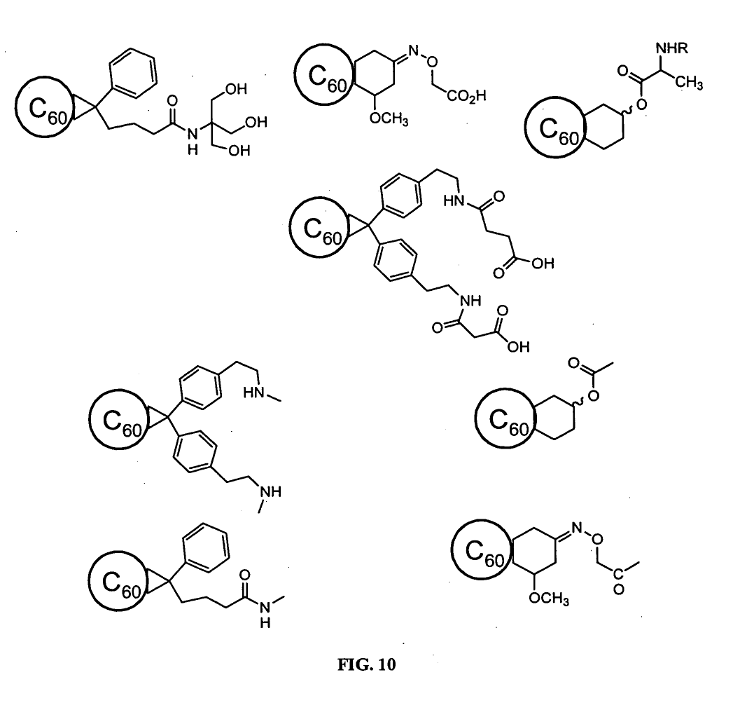

Alternatively, endohedral fullerenes, i.e., fullerene with metal atoms encapsulated within the fullerene, can be used in both X-ray and magnetic resonance (MR) imaging procedures. Preferred metals for use in endohedral fullerenes include, but are not limited to, holmium, gadolinium, technetium, rhodium. The solubility of the endohedral fullerenes can be enhanced by the addition of polar groups, such as polyamine or polyhydroxy groups. Exemplary polar groups are illustrated in the first four compounds of FIG. 10, while the last four compounds illustrate sites where an anticoagulant moiety could be attached to the fullerene scaffold.

The globular shape of a fullerene containing contrast media will reduce viscosity. In addition, the fullerene core masks one side of the tri-iodinated benzene ring, thus blocking hydrophobic interactions with blood plasma proteins for increased tolerability. Fullerene compounds have been shown to rapidly distribute into tissues. Thus a fullerene-containing contrast can be used to image internal tissues as well as cavities within the body.

b. Electron-Dense Heavy Metals

While not commonly used as X-ray contrast agents, electron-dense heavy metals are contemplated as imaging moieties in the anticoagulant contrast agents of the present invention. The current use of iodinated X-ray contrast agents is largely based on their known safety and low cost of iodine rather than its optimal efficiency as an X-ray attenuator. A new X-ray contrast agent based on heavy moieties would have two advantages; 1) a higher intrinsic contrast and 2) a lower radiation exposure to patients.

For a heavy metal-based X-ray contrast agent to be competitive with water-soluble iodinated agents for general use it preferably possesses the following attributes: 1) high water solubility, 2) stability under physiologic conditions, 3) comparative pharmacokinetic profile, 4) complete excretion, 5) high safety and 6) low osmolality and viscosity. Likely acute and long-term toxicities and the inability of the human body to completely excrete heavy metals limits the usefulness of simple heavy metal salts or metal particulates as intravascular X-ray agents. The present invention avoids these problems by attaching the heavy metal imaging moieties to a supporting organic scaffold moiety as described herein. Preferred heavy metals for use in contrast agents include, but are not limited to, the lanthanide metals and Hf, Ta, W, Re and Bi.

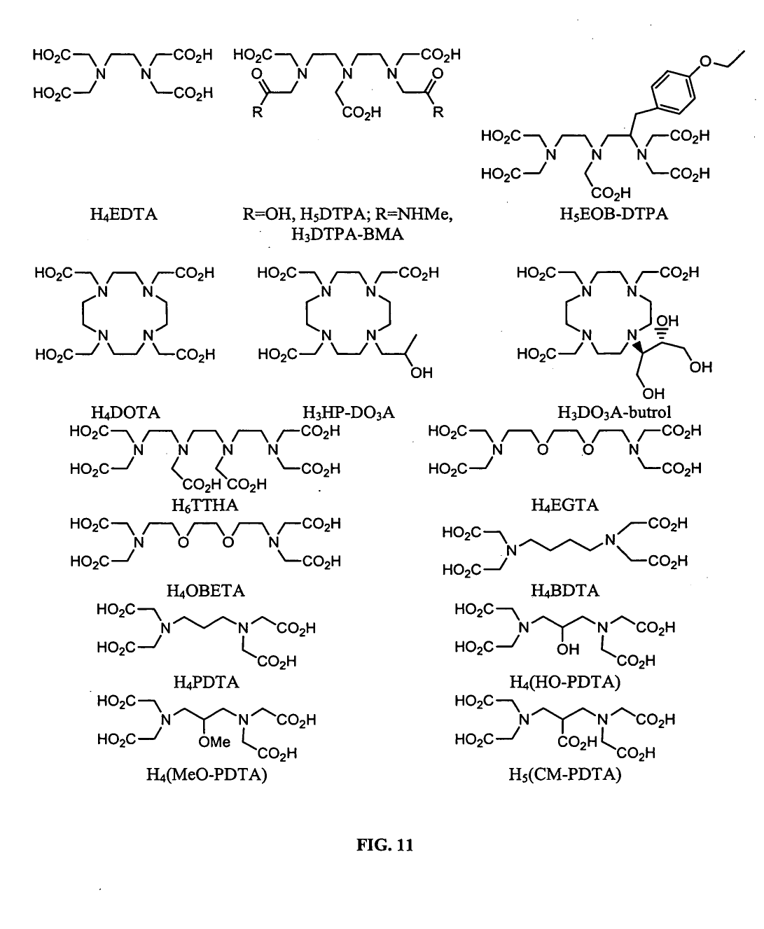

A preferred scaffold moiety for use in the present invention is a metal chelator. A metal-chelator complex is a coordination compound of a metal ion with a chelating agent (often an organic ligand). The chelating agent has at least two functional groups which donate a pair of electrons to the metal, such as O, NH2 or COO⁻. The chelating agent alters the behavior in biological systems of the metal ions it binds. This includes the biodistribution, excretion profile and toxicity of the chelated metal. Magnetic resonance contrast agents have been successfully developed over the past two decades utilizing either linear or cyclic polyaminopolycarboxylate ligands. Examples of commercially available MR contrast agents include Omniscan (Gadodiamide), Optimark (Gadoversetamide), Magnevist (Gadopentetate dimeglumine) and Prohance (Gadoteridol). Common chelators are illustrated in FIG. 11

Metal cluster compounds contain two or more metal atoms and involve substantial metal-metal bonding and have two advantages over iodinated benzene based compounds. First, the highly concentrated solutions provide greater radiodensity. Second, these greater radiodensity results in a greatly reduced radiation dose to the patient. The metal clusters can be chelated with polyaminopolycarboxylate ligands as illustrated in FIG. 11 (Yu et al., Inorg. Chem. 40:1576-1581 (2001). The anticoagulant moiety can be attached to the ligand through any functional group that is not required for metal coordination. In a preferred embodiment, the anticoagulant contrast media comprises a tungsten cluster compound.

Extensive reviews on the preparation, reaction chemistry and physical properties of organobismuth compounds are readily available. See, e.g., Yu and Watson, Metal-Based X-ray Contrast Media. Chem Rev 99:2353-77 (1999). In addition to the advantages inherent to all the heavy metal complexes, organobismuth compounds have two distinct advantages; 1) covalent bond between bismuth and aromatic carbons are very strong and stable similar to a carbon-iodine bond and 2) organobismuth compounds are usually nonionic. One preferred embodiment of the invention comprises water-soluble triaryl bismuth moieties as the scaffold and imaging moieties. The organic bismuth derivative, preferably, has the structure: 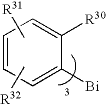
wherein
R³⁰ is ANR³³R³⁴, wherein A is —SO₂— or —C(O)—, R³³ is C3-C6 alkyl having from 2 to 5 hydroxyl groups, which optionally are protected, or together with R³⁴ forms a C2-C6 alkylene having from 1 to 6 hydroxyl groups, which optionally are protected, and R³⁴ is hydrogen, C1-C6 alkyl or C1-C5 acyl having from 1 to 5 hydroxyl group, which optionally are protected or together with R³³ forms a C2-C6 alkylene having from 1 to 6 hydroxyl groups, which optionally are protected;
R³¹ is hydrogen, C1-C4 alkyl, C1-C4 alkylcarbonyl, nitro, cyano or ANR³³R³⁴, wherein A is —SO₂— or —C(O), R³³ is C3-C6 alkyl having from 2 to 5 hydroxyl groups, which optionally are protected, or together with R³⁴ forms a C2-C6 alkylene having from 1 to 6 hydroxyl groups, which optionally are protected, and R³⁴ is hydrogen, C1-C6 alkyl or C1-C5 acyl having from 1 to 5 hydroxyl group, which optionally are protected or together with R³³ forms a C2-C6 alkylene having from 1 to 6 hydroxyl groups, which optionally are protected;
R³² is hydrogen, C1-C4 alkyl, C1-C4 alkylcarbonyl, nitro, cyano or ANR³³R³⁴, wherein A is —SO₂— or —C(O), R³³ is C3-C6 alkyl having from 2 to 5 hydroxyl groups, which optionally are protected, or together with R³⁴ forms a C2-C6 alkylene having from 1 to 6 hydroxyl groups, which optionally are protected, and R³⁴ is hydrogen, C1-C6 alkyl or C1-C5 acyl having from 1 to 5 hydroxyl group, which optionally are protected or together with R³³ forms a C2-C6 alkylene having from 1 to 6 hydroxyl groups, which optionally are protected.

Exemplary organic bismuth contrast agents include:

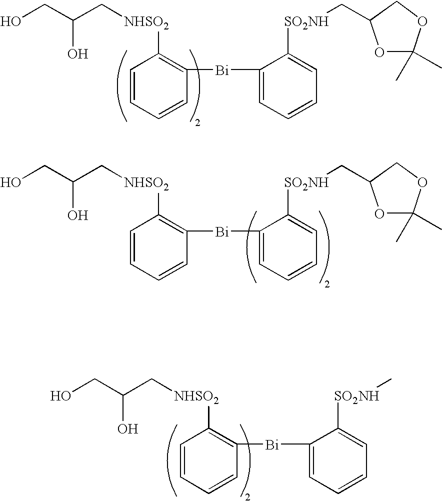

The imaging and organic scaffold moieties are attached to the anticoagulant moiety using standard chemical methods.

D. Anticoagulant Contrast Media

An anticoagulant contrast media comprising the anticoagulant contrast agent preferably will have physiological osmolality to improve patient's tolerance and decreased viscosity to minimize renal complications. Unit doses or multiple dose forms are contemplated, each offering advantages in certain clinical settings. The unit dose would contain a predetermined quantity of anticoagulant contrast agent calculated to produce the desired effect(s) of aiding the visualization of internal organs and/or structures. The multiple dose form may be particularly useful when multiple scans or scans of different areas of the body are required to achieve the desired ends. Administration can be systemic or local. Either of these dosing forms may have specifications that are dictated by or directly dependent upon the unique characteristic of the particular compound, the particular image to be achieved, and any limitations inherent in the art of preparing the particular compound for visualization of particular internal structures or organs.

Suitable formulations are prepared in accordance with standard formulating techniques available that match the characteristics of the contrast agent to the carriers available for formulating an appropriate contrast media. For example, the compounds of this invention can be employed in mixture with conventional excipients, i.e., pharmaceutically acceptable organic or inorganic carrier substances suitable for parenteral or enteral application which do not deleteriously react with the active compounds.

Also included are the pharmaceutically acceptable salts of the anticoagulant contrast agents. Pharmaceutically acceptable salts are those salts formed by reacting an organic or inorganic acid with the contrast agent where there is a reactive base (e.g., an available nitrogen). Suitable salts include, but are not limited to, the acetate, hydrochloride, sulfate, phosphate. Pharmaceutically acceptably salt also can be formed by reacting an organic or inorganic base with the contrast agent where there is a reactive acid, e.g., a carboxylic acid group. Other suitable salts will be apparent to one of skill in the art by consulting standard sources such as Remington's Pharmaceutical Sciences, 18^{th} Ed., © 1990, in particular, Chapter 66: Pharmaceutical Necessities, pp. 1286-1329.

A compound may be administered parenterally, e.g., intravenously, intramuscularly, intrathecally, subcutaneously, or interperitonieally. The carrier can be a solvent or a dispersive medium containing, for example, various polar or non-polar solvents, suitable mixtures thereof, or oils. Solutions of the compound may be prepared in suitable diluents such as water, ethanol, glycerol, liquid polyethylene glycol(s), various oils, and/or mixtures thereof, and others known to those skilled in the art. As used herein “carrier” or “excipient” means a pharmaceutically acceptable carrier or excipient and includes, but is not limited to, any and all solvents, dispersive agents or media, coating(s), antimicrobial agents, iso/hypo/hypertonic agents, absorption-modifying agents. The use of such substances and the agents for pharmaceutical substances is well known in the art. Except insofar as any conventional media or agent is incompatible with the anticoagulant contrast agent, use in the anticoagulant contrast media of the invention is contemplated. Moreover, other or supplementary active ingredients can also be incorporated into the final anticoagulant contrast media.

In one embodiment, anticoagulant contrast media are formulated in accordance with routine procedures as a pharmaceutical composition adapted for intravenous administration to human beings. Typically, compositions for intravenous administration are solutions in sterile isotonic aqueous buffer. For injection, a compound may be formulated in aqueous solutions, preferably, in physiologically compatible buffers such as Hanks' solution, Ringer's solution, or physiological saline buffer. The solution may contain formulatory agents such as suspending, stabilizing and/or dispersing agents. When necessary, the anticoagulant contrast media may also include a solubilizing agent. Anticoagulant contrast media for intravenous administration may optionally include a local anesthetic such as lignocaine to ease pain at the site of the injection. Generally, the ingredients are supplied either separately or mixed together in unit dosage form, for example, as a lyophilized powder or water free concentrate in a hermetically sealed container such as an ampoule or sachette indicating the quantity of active agent. When an anticoagulant contrast agent is administered by infusion, it can be dispensed, for example, with an infusion bottle containing sterile pharmaceutical grade water or saline. When a compound is administered by injection, an ampoule of sterile water for injection or saline can be provided so that the ingredients may be mixed prior to administration.

The pharmaceutical forms suitable for injectable use include sterile solutions, dispersions, emulsions, and sterile powders. The final form must be stable under conditions of manufacture and storage. Furthermore, the final pharmaceutical form must be protected against contamination and must, therefore, be able to inhibit the growth of microorganisms such as bacteria or fungi. A single intravenous or intraperitoneal dose can be administered. Alternatively, a slow long term infusion or multiple short term infusions may be utilized, typically lasting from 20 minutes to 2 hours.

Sterile, injectable solutions are prepared by incorporating an anticoagulant contrast agent in the required amount into one or more appropriate solvents to which other ingredients, listed above or known to those skilled in the art, may be added as required. Sterile injectable solutions are prepared by incorporating the anticoagulant contrast agent in the required amount in the appropriate solvent with various other ingredients as required. Sterilizing procedures, such as filtration, then follow. Typically, dispersions are made by incorporating the anticoagulant contrast agent into a sterile vehicle which also contains the dispersion medium and the required other ingredients as indicated above. In the case of a sterile powder, the preferred methods include vacuum drying or freeze drying to which any required ingredients are added.

In all cases the final form, as noted, must be sterile and must also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

Prevention or inhibition of growth of microorganisms may be achieved through the addition of one or more antimicrobial agents such as chlorobutanol, ascorbic acid, parabens, thermerosal, or the like. It may also be preferable to include agents that alter the tonicity such as sugars or salts.

In certain embodiments, an anticoagulant contrast agent and/or media can be used in combination with at least one therapeutic agent. In one embodiment, the anticoagulant contrast agent and/or media is administered concurrently with the administration of a therapeutic agent. In another embodiment, the anticoagulant contrast agent and/or media is administered prior or subsequent to administration of a therapeutic agent.

Examples of therapeutic agents which can be used with the contrast agents and media of the invention include, but are not limited to, anti-emetic, tranquilizing agents or muscle relaxants.

Examples of agents for controlling nausea (antiemetics) include, but are not limited to, benzquinamide hydrochloride, chlorpromazine, buclizine hydrochloride, cyclizine, cyclizine hydrochloride, cyclizine lactate, dimenhydrinate, diphenhydramine hydrochloride, diphenidol, hydrochloride, dronabinol, meclizine hydrochloride, pheniramine male ate, metoclopramide hydrochloride, prochlorperazine, prochlorperazine edisylate, prochlorperazine maleate, scopolamine hydrobromide, thiethylperazine malate, thiethylperazine maleate, triflupromazine, and trimethobenzamide hydrochloride.

Examples of tranquilizing agents include, but are not limited to, benzodiazepines (e.g., alprozam, chlordiazepoxide, chlordiazepoxide hydrochloride, chlorazepate dipotassium, diazepam, flurazepam hydrochloride, halazepam, lorazepam, oxazepam, prazepam, temazepam, triazolam, flunitrazepam and quazepam), barbiturates (e.g., amobarbital, amobarbital sodium, butabarbital sodium, mephobarbital, pentoparbital, pentoparbital sodium, phenobarbital, Phenobarbital sodium, secobarbital, secobarbital sodium, talbutal, and aprobarbital), buspirone hydrochloride, chloral hydrate, disulfiram, ethchlorvynol, ethinamate, glutethimide, hydroxyzine hydrochloride, hydroxyzine pamoate, meprobamate, methprylon, paraldehyde, chlormezanone and propiomazine hydrochloride.

Examples of muscle relaxants include, but are not limited to, nicotinic agonists (e.g., atracurium besylate, dantrolene sodium, hexafluorenium bromide, metocurine iodide, succinylcholine chloride, tubocurarine chloride, vecuronium bromide), and centrally acting muscle relaxants (e.g., baclofen, carisoprodol, chlorzoxone, cyclobenzaprine hydrochloride, methocarbamol).

Trace quantities of certain cations (Fe, Cu) are able to catalyze the deiodination of iodinated benzene X-ray agents during the formulation and sterilization process. Therefore, anticoagulant contrast media that have a contrast agent comprising an iodinated benzene ring preferably will include a sequestering agent or weak amino buffer. Addition of small quantities of sequestering agents (such as calcium di-sodium edetate or similar agent) has proved to prevent catalytic deiodination. Addition of small quantities of weak amino buffers (such as Tris) can protect deiodination for nonionic iodinated benzene agents. Eloy, et al., Contrast Media for Angiography: Physiochemical Properties, Pharmacokinetics and Biocompatibility, Clinical Materials 7:89-197 (1991).

The concentration of the novel contrast agent of this invention in media depends on the particular diagnostic method involved. The preferred concentrations and doses of the compounds of this invention, e.g., for X-ray diagnoses, are concentrations of 50-500 mg of iodine per ml and doses of 10-500 ml. Concentrations of 100-350 mg of iodine per ml are especially preferred. Heavy metal contrast agents, such as gadolinium MR contrast agents, are administered at concentrations of 250-610 mg of chelated heavy metal per ml and at doses from 0.1-0.3 mmol/kg body weight.

E. Methods of Imaging Internal Structures and Organs

The anticoagulant contrast media of the invention are suitable for use in all indications in which current contrast media are used. These indications include coronary arteriography with or without left ventriculography, peripheral arteriography, aortography, visceral arteriography, cerebral angiography, intra-arterial digital subtraction angiography, intravenous digital subtraction angiography, peripheral venography, excretory urography, contrast enhancement of computed tomographic head imaging and body imaging, arthrography and hysterosalpingography. The anticoagulant contrast agents of the present invention are particularly useful in the visualization of the cardiovascular system and for cerebral angiography.

| Route | | | | |
|---|---|---|---|---|
| Adminis- | Imaging | Scaffold | Imaging | |
| tered | Moiety | Moiety | Procedure | Internal Structure |
| Intravenous | heavy | chelators, | MRI | central nervous system |
| | metals | clusters, | | liver |
| | | cages | | body (noncardiac |
| | | | | intrathoracic, intra- |
| | | | | abdominal, pelvic, |
| | | | | retroperitoneal) |
| Intravenous | heavy | chelators, | X-ray | blood pool |
| | metals | clusters, | | cardiac |
| | | cages | | |
| Intra-arterial | iodine, | benzene, | X-ray | blood pool |
| | bromine | dicarboranes, | | cardiac |
| | | cages | | cerebral angiography |
| | | | | excretory urography |
| | | | | CT head imaging |
| | | | | CT body imaging |
| | | | | arthrography |
| | | | | hysterosalpinography |
| Intravenous | iodine, | benzene, | X-ray | blood pool |
| | bromine | dicarboranes, | | cardiac |
| | | cages | | CECT of head or body |
| | | | | excretory urography |
| | | | | venography |
| | | | | cerebral angiography |
| | | | | arthrography |
| | | | | hysterosalpinography |
| Intrathecally | iodine, | benzene, | X-ray | CECT of head |
| | bromine | dicarboranes, | | cerebral angiography |
| | | cage | | |

The anticoagulant contrast media of the invention are aqueous solutions containing, for example, 15 g and more of the anticoagulant contrast agent per 100 ml of solution, equivalent, e.g., to 140 to approximately 350 mg iodine per ml. The more concentrated solutions are generally preferred, and they are applied in a manner generally known and selected according to the body cavity that will be visualized. In vasography, the solutions are injected or infused into the vessels, particularly the blood vessels. Intravenous injection is resorted to in urography. For myelography and radiculography, the solutions are instilled after lumbar or suoccipital puncture. The amounts of solution necessary vary depending on the agent used and its concentration, but will generally fall in the 0.1-100 ml range for ease of measurement, handling and injection. Such amounts of solution have been used in the past. For example (in accordance with the instructions provided in the Omnipaque package insert), the usual recommended total doses of Omnipaque for use in myelography in adults are 1.2 g iodine to 3.06 g iodine and in pediatrics, 0.36 g iodine to 2.94 g iodine. The usual recommended total dose of Omnipaque 350 for use in ventriculography for adults is 40 ml with a range of 30 to 60 ml and for pediatrics is 1.25 ml/kg of body weight with a range of 1.0 ml/kg to 1.5 ml/kg

Considerable guidance to dosage and usage is available from use of other contrast agents in the past. For MR diagnostic evaluation, for example, one recent patent has suggested that the diagnostic agent, if in solution, suspension or dispersion form, will generally contain the metal chelate at a concentration in the range of 1 micromol to 1.5 mol per liter, preferably 0.1 to 700 mM. The patent went further to say that the diagnostic agent may be supplied in a more concentrated form for dilution prior to administration. Exemplary amounts are suggested as being from 10⁻³ to 3 mmol of the metal species per kilogram of body weight, for example about 0.1 mmol of a lanthanide (e.g. Dy or Gd)/kg body weight. See Hollister et al., U.S. Pat. No. 5,801,228, “Polymeric Contrast Agents for Medical Imaging.”

For examples of X-ray examination, where the dose of the contrast agent should generally be higher, see Berg et al., U.S. Pat. No. 5,198,208, “Aminopolycarboxylic Acids and Derivatives Thereof,” which suggest that for for MR diagnostic evaluation, the diagnostic agent, if in solution, suspension or dispersion form, will generally contain the metal chelate at a concentration in the range of 1 micromol to 1.5 mol per liter, preferably 0.1 to 700 mM. This patent goes further to say that the diagnostic agent may be supplied in a more concentrated form for dilution prior to administration, such as from 10⁻⁴ to 1 mmol of the metal species per kilogram of bodyweight.

Two other patents, namely Woulfe, U.S. Pat. No. 5,961,953, “Magnetic Resonance Blood Pool Agents” and Weber, U.S. Pat. No. 5,130,120, “Paramagnetic DTPA and EDTA Alkoxyalkylamide Complexes as MRI Agents,” offer additional suggestions, stating that, in general, parenteral dosages will range from about 0.001 to about 1.0 mmol of paramagnetic ion complex per kg of patient body weight. Preferred parenteral dosages stated in these last two patents range from about 0.01 to about 0.5 mmol of paramagnetic ion complex per kg of patient body weight, and enteral dosages generally range from about 0.5 to about 100 mmol, preferably form about 1.0 to 10 to about 10 mmol, more preferably from about 1.0 to about 20.0 mmol of paramagnetic ion complex per kg of patient body weight.

Other examples of useful paramagnetic solutions are available from commercial gadolinium solutions (Gd MW=157). Omniscan is 287 mg Omniscan/ml, MW 573, 27% Gd; Gadovist is 604 mg Gadovist/ml, MW 604, 25% Gd; Gadoteridol is 279 mg gadoteridol/ml, MW 558, 28%. See also Hollister et al., U.S. Pat. No. 5,801,228, “Polymeric Contrast Agents for Medical Imaging,” which desribes polymeric contrast agents containing gadolimium in the range of 19-30% Gd by weight.

The precise method and details of application depend on the organ which is to be visualized and can be determined by fully conventional considerations, e.g., in analogy with conventional media such as those described in U.S. Pat. No. 4,264,572.

### V. EXAMPLES

The following examples are provided as a guide for a practitioner of ordinary skill in the art. The examples should not be construed as limiting the invention, as the examples merely provide specific methodology useful in understanding and practicing an embodiment of the invention.

### A. Example 1

### Synthesis of Compound I (Monomeric P1)

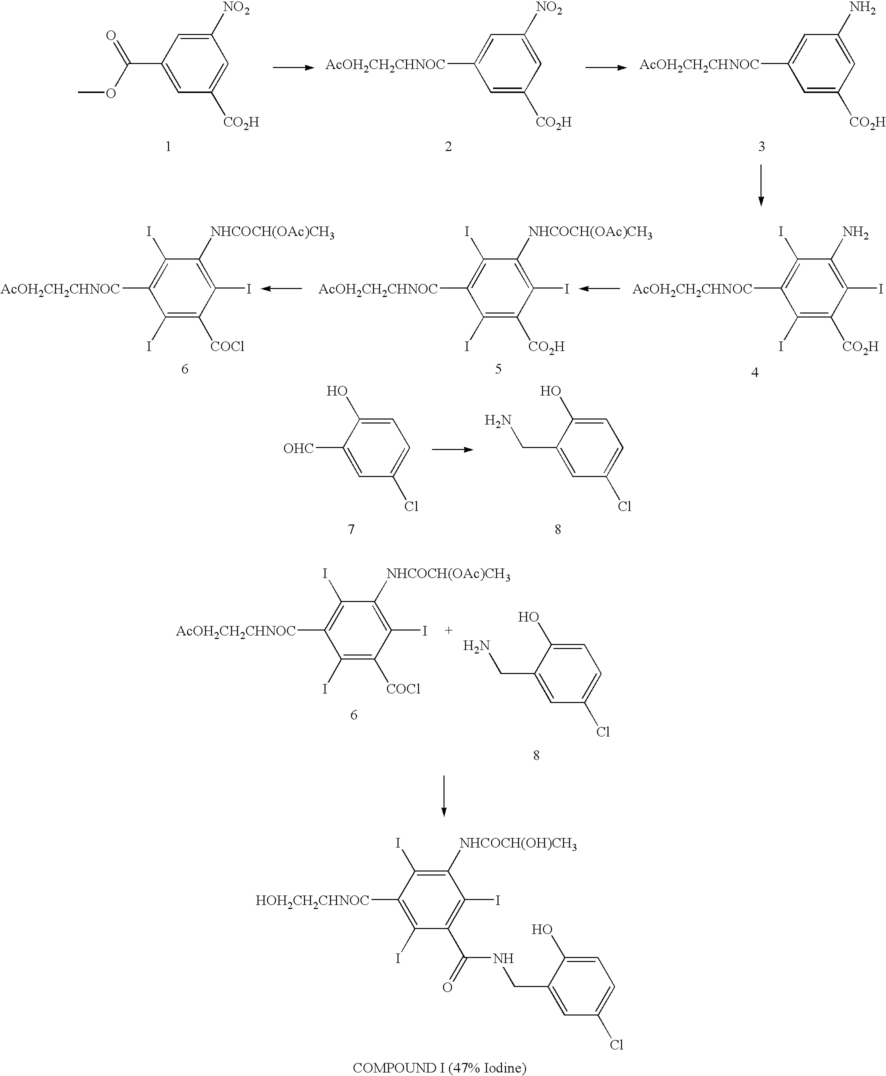

Treatment of the 5-nitro-isophthalic acid monomethyl ester (1) with an amine alcohol results in the formation of a 5-nitro-N-alkylisophthalamic acid (2). Reduction of the nitro group, by catalytic hydrogenation yields the corresponding 5-amino-N-hydroxylalkylisophthalamic acid (3). Catalytic hydrogenation of the 5-nitro-N-hydroxylalkylisophthalamic acid is carried out on the free acid, dissolved in ethanol. The 5-amino-N-hydroxylalkylisophthalamic acid (3) is converted to the corresponding triiodo compound (4) by iodination with I₂ in the presence of K₁ and C₂H₅NH₂. Treatment of the iodinated amino compound (4) with an hydroxylalkanoic acid anhydride in the presence of sulfuric acid results in the formation of a 5-alkanamido-2,4,6-triiodo-N-hydroxylalkylisophthalamic acid (5). Conversion of this acid to an acid halide (6) is accomplished with thionyl chloride. The 2-hydroxy-5-chlorobenzylamine (8) is prepared in two steps from the aldehyde (7) via reduction of the oxime as described in Tucker et al. Design and Synthesis of a Series of Potent and Orally Bioavailable Noncovalent Thrombin Inhibitors That Utilize Nonbasic Groups in the P1 Position. J Med Chem 41:3210-19 (1998). The acid chloride (6) is condensed with the primary amine (8) resulting in the formation of Compound I.

### B. Example 2

### Synthesis of Compound II (Dimeric P1)

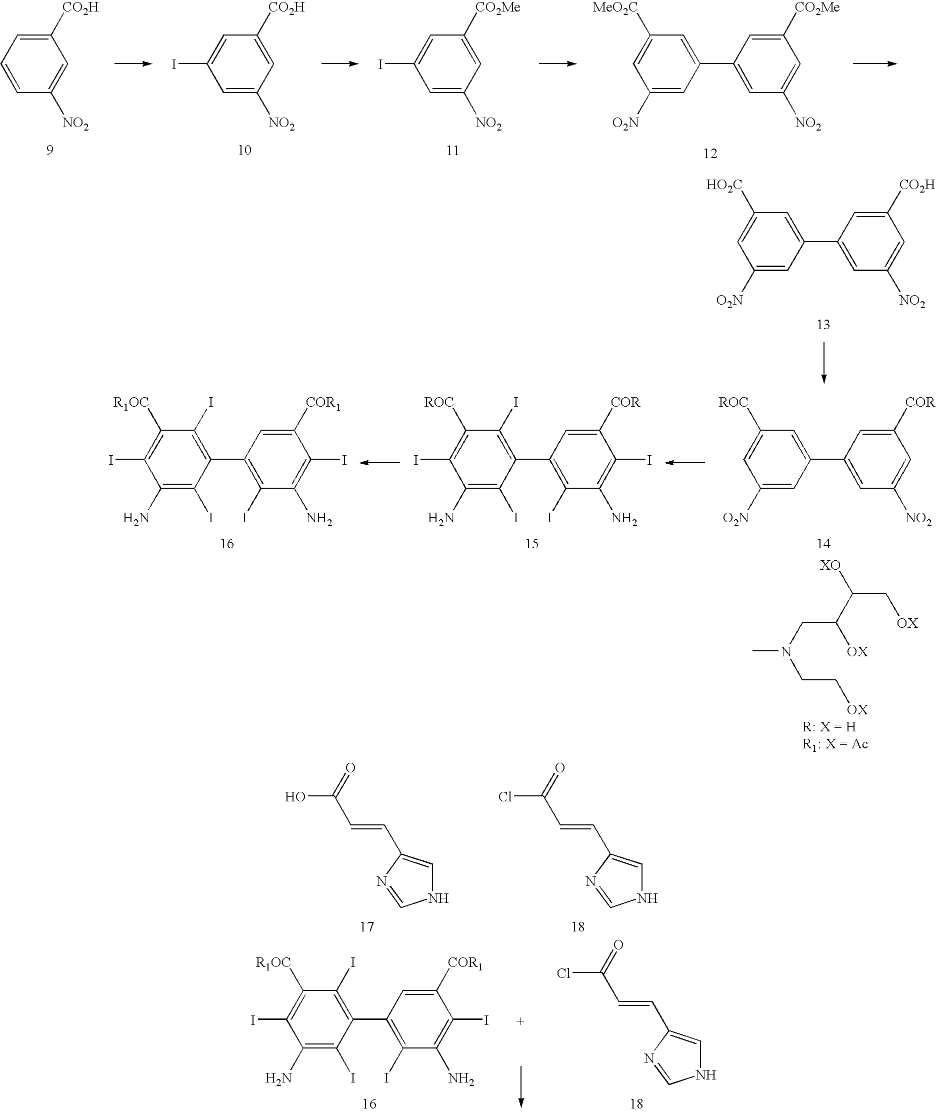

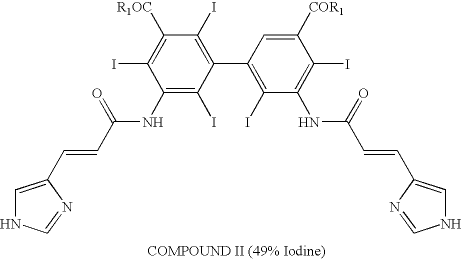

The 3-nitro-benzoic acid (9) is converted to the corresponding 5-nitro-3-iodoisophthalic acid (10) by iodination with I₂ in the presence of K₁ and C₂H₅NH₂. Esterification of the mono-iodinated compound results in the formation of the methyl ester (11). The methyl ester dimerizes to the dinitrobiphenyl diester (12), utilizing Ullman's method of heating the methyl ester in the presence of copper (Fanta PE. The Ullman Synthesis of Biaryls. Chem Rev 1964; 64:613-32). Saponification of the dinitrobiphenyl diester results in the formation of the corresponding diacid (13). Conversion of the diacid to an acid dichloride with thionyl chloride, followed by introduction of hydrophilic side chains by amidification results in the dinitrobiphenyldiamide (14). The dinitrobiphenyldiamide is reduced by catalytic hydrogenation producing the diaminobiphenyldiamide. The diaminobiphenyldiamide is converted to the corresponding triiodo compound (15) by iodination with I₂ in the presence of K₁ and C₂H₅NH₂, with I₂ dissolved in oleum. Acetylation of the hydroxyl groups produces the protected diaminobiphenyldiamide (16). Urocanic acid (17) is converted to the corresponding acyl halide (18) utilizing thionyl chloride. Acylation of the two aminoaromatic groups (16) with the acid chloride (18), followed by removal of protection groups produces Compound II.

### C. Example 3

### Synthesis of Compound III (Monomeric P2-P1)

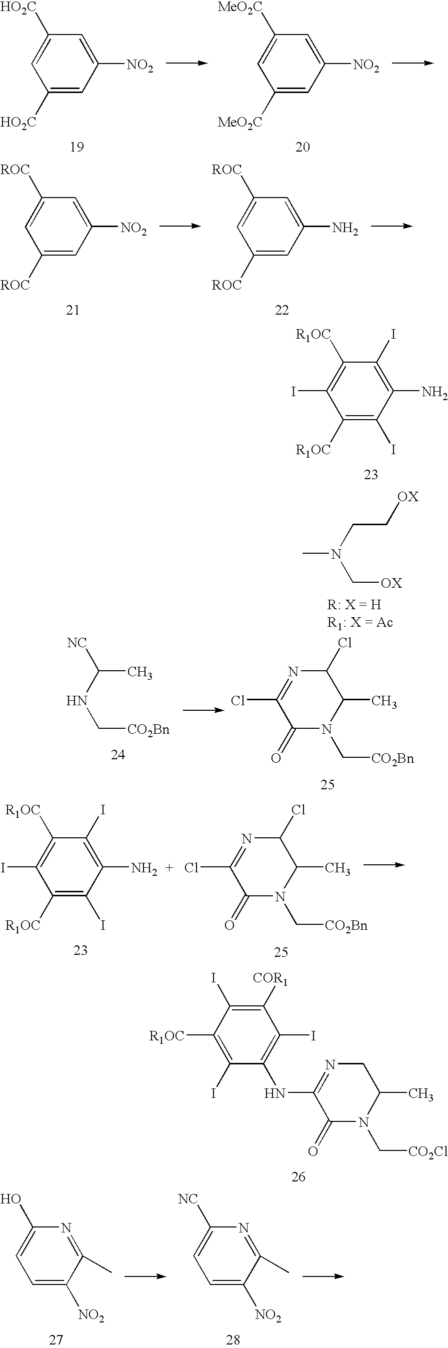

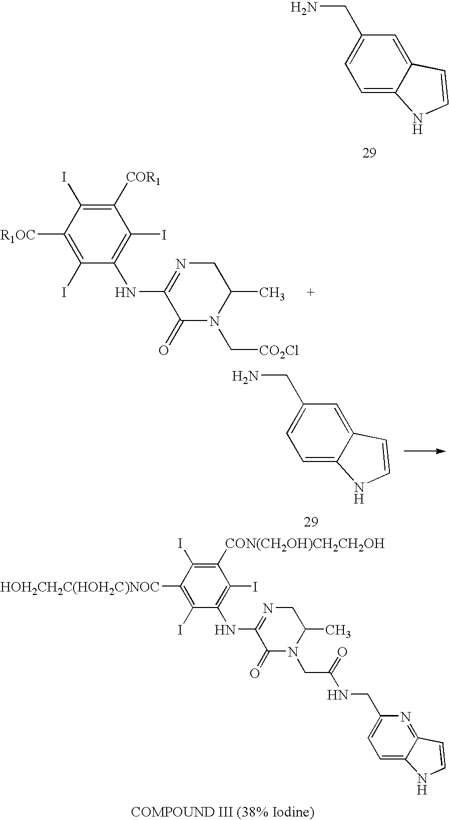

The 5-nitro-isophthalic acid (19) in the presence of methanol is converted to the corresponding dimethyl ester (20). The hydrophilic side chains are introduced by amidation of the dimethyl ester with a hydroxylamine resulting in nitro-isophthalamide (21). The nitro-isophthalamide is reduced by catalytic hydrogenation to the corresponding amino-isophthalamide (22). The nitro groups may also be reduced by metallic zinc in an aqueous solution of ammonium chloride. The amino-isophthalamide is converted to the corresponding triiodo compound by iodination with an I₂/H₅IO₆ mixture. Acetylation of the hydroxyl groups produces the protected triiodo compound 23, as described in Priebe et al. Synthesis and Characterization of Iodixanol. Acta Radiologica 36(S399):21-31 (1995). The aminonitrile 24 is prepared as an oil from the free base of glycine benzyl ester utilizing the procedure of Gibson (Leblanc and Gibson, Synthesis of α-Aminonitriles by Self-catalyzed, Stoichiometric Reaction of Primary Amines, Aldehydes, and Trimethylsilyl Cyanide. Tetrahedron Lett 33:6295-8 (1992)). Subjection of the salt of the aminonitrile to the Hoomaert conditions (COCl₂, o-C₆H₄Cl₂, 100° C., 15 h) produces the dichloropyrazinone 25. Displacement of the 3-chloro group of the dichloropyrazinone occurs upon addition of triiodoamine compound, resulting in the aminopyrazinone. The aminopyrazinone is hydrolyzed to remove the benzyl group followed by dechlorination by palladium-catalyzed hydrogenolysis, resulting in the formation of the acid. Conversion of the acid to the acid halide 26 occurs by addition of thionyl chloride (Sanderson et al. Efficacious, Orally Bioavailable Thrombin Inhibitors Based on 3-Aminopyridinone or 3-Aminopyrazinone Acetamide Peptidomimetic Templates. J Med Chem 41:4466-74 (1998)). The synthesis of the 4-azaindole amine 29 starts from commercially available 6-methyl-5-nitropyridin-2-ol (27). Conversion to the nitrile 28 in two steps (first with POBr₃, (CHCl₂)₂, reflux, 4 h followed by Zn(CN)₂, (Ph₃P)₄Pd, DMF, 80° C., 5 h) followed by a BatchoLeimgruber procedure with concomitant reduction of the nitrile results in the 5-aminomethyl-4-azaindole 29 (Sanderson et al. Azaindoles: Moderately Basic P1 Groups for Enhancing the Selectivity of Thrombin Inhibitors. Bioorg Med Chem Lett 13:795-8 (2003)). Coupling of the acid halide 26 with the 5-aminomethyl-4-azaindole 29, followed by removal of the hydroxyl protecting groups results in Compound III.

### D. Example 4

### Synthesis of Compound IV (Monomeric P2-P1)

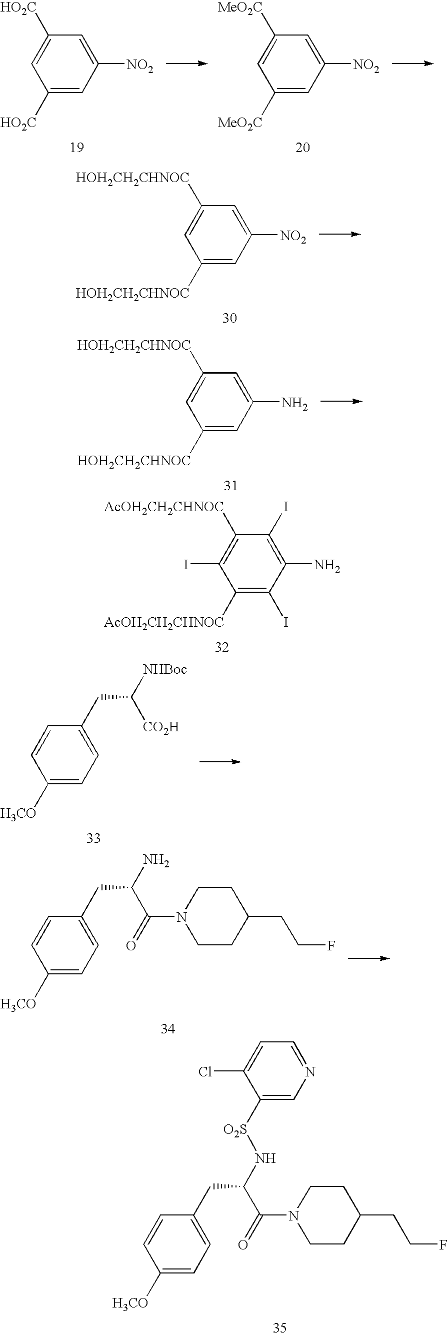

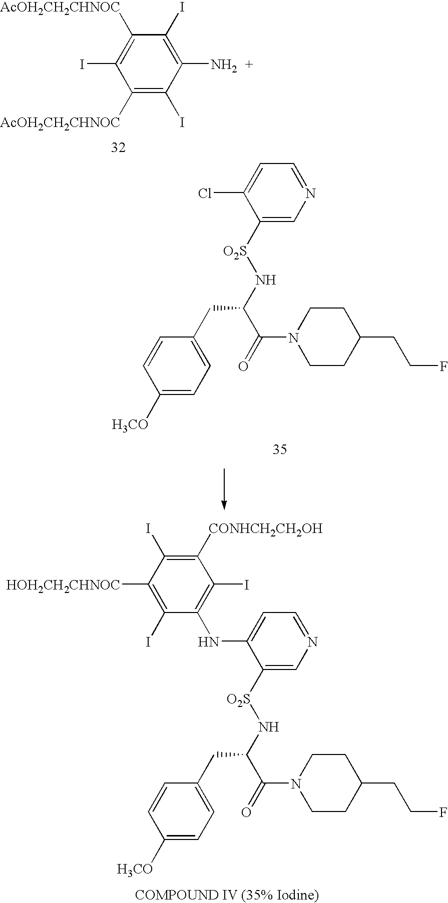

The 5-nitro-isophthalic acid 19 in the presence of methanol is converted to the corresponding dimethyl ester 20. Hydrophilic side chains are introduced by amidation of the dimethyl ester with a hydroxylamine to form nitro-isophthalamide 30. The nitro-isophthalamide is reduced by catalytic hydrogenation to amino-isophthalamide 31. The amino-isophthalamide is converted to the corresponding triiodo compound by iodination with potassium iododichloride (KICl₂), followed by acetylation of the hydroxyl groups to produce the protected triiodo compound 32. 4-Fluoroethyl piperidine is condensed with a Boc protected amino acid 33 using 2-(1H-benzatriazole-1-yl)-1,1,3,3 tetramethylammonium tetraflouroborate, followed by removal of the Boc protection group by treatment with 6M HCl/ethanol to produce the amine 34. The amine is coupled to chloropyridine-3-sulfonyl chloride to produce the chloropyridyl derivative 35. Substitution of the 4-chloro group 35 with excess triiodoamine 32, followed by removal of the hydroxyl protecting groups affords Compound IV.

### E. Example 5

### Synthesis of Compound V (Monomeric P3-P2-P1)

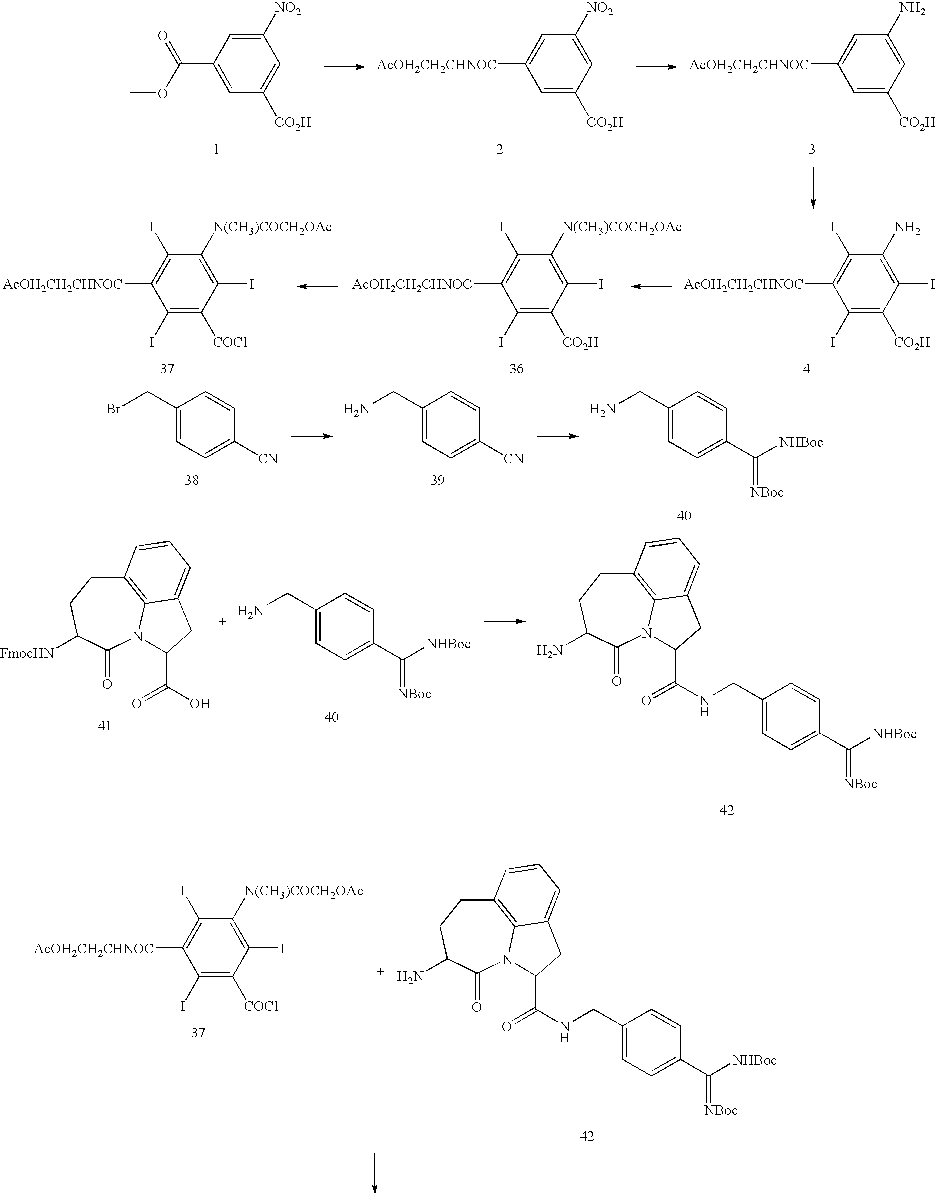 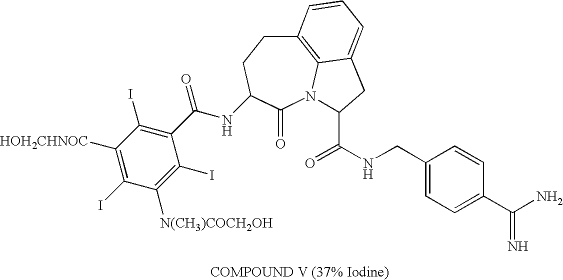 Treatment of the 5-nitro-isophthalic acid monomethyl ester (1) with an amine alcohol results in the formation of a 5-nitro-N-alkylisophthalamic acid (2). Reduction of the nitro group, by catalytic hydrogenation, yields the corresponding 5-amino-N-hydroxylalkylisophthalamic acid (3). Catalytic hydrogenation of the 5-nitro-N-hydroxylalkylisophthalamic acid is carried out on the free acid, dissolved in ethanol. The 5-amino-N-hydroxylalkylisophthalamic acid (3) is converted to the corresponding triiodo compound (4) by iodination with iodine monochloride. Treatment of the iodinated amino compound (4) with acyl halide alcohol in the presence of sulfuric, results in the formation of a 5-alkanamido-2,4,6-triiodo-N-hydroxylalkylisophthalamic acid (36). Conversion of this acid to an acid halide (37) is accomplished with thionyl chloride (Hoey, U.S. Pat. No. 3,145,197).

Treatment of p-cyanobenzyl bromide (38) with sodium azide followed by hydrogenation with Pd/C results in the formation of the p-cyanobenzyl amine (39). The p-cyanobenzyl amine is treated with a hydroxylamine, followed by hydrogenation with Pd/C to produce the Boc protected 4-aminomethylbenzamidine (40). Amide coupling of Fmoc-aminohexanhydroazepinoindole-4-one-2-carboxylic acid with the amino benzamidine, followed by removal of the FMOc protecting group produces the free amine compound (42). (Ho, et al., Bioorg Med Chem Lett 2002; 12:743-8).

The acid chloride (37) is condensed with the primary amine (42), followed by the removal of hydroxyl protecting groups results in the formation of Compound V.

### F. Example 6

### Synthesis of Compound VI (Monomeric P3-P2-P1)

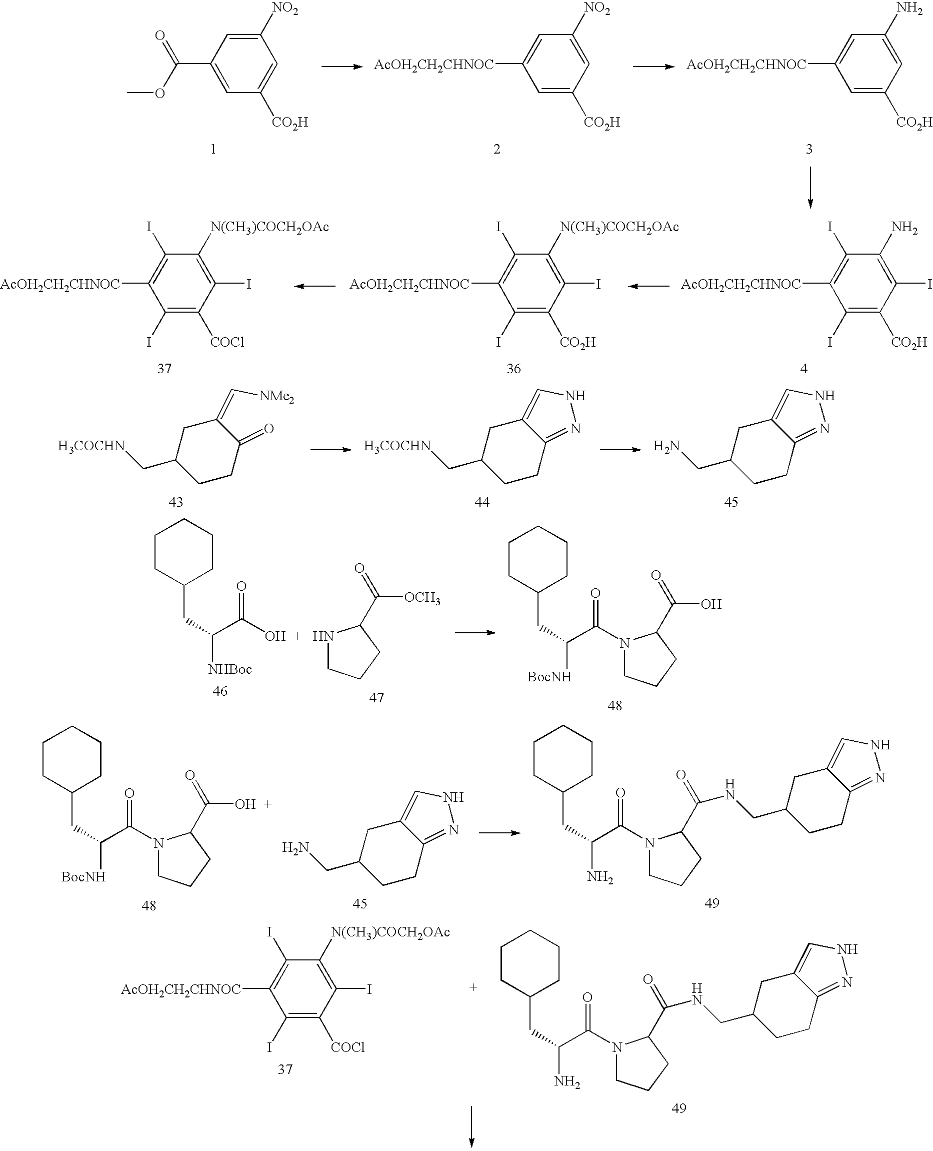

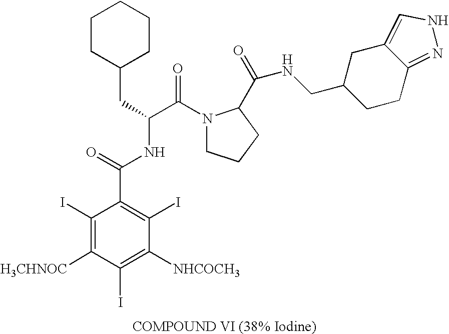

Treatment of the 5-nitro-isophthalic acid monomethyl ester (1) with an amine alcohol results in the formation of a 5-nitro-N-alkylisophthalamic acid (2). Reduction of the nitro group, by catalytic hydrogenation, yields the corresponding 5-amino-N-hydroxylalkylisophthalamic acid (3). Catalytic hydrogenation of the 5-nitro-N-hydroxylalkylisophthalamic acid is carried out on the free acid, dissolved in ethanol. The 5-amino-N-hydroxylalkylisophthalamic acid (3) is converted to the corresponding triiodo compound (4) by iodination with iodine monochloride. Treatment of the iodinated amino compound (4) with acyl halide alcohol in the presence of sulfuric, results in the formation of a 5-alkanamido-2,4,6-triiodo-N-hydroxylalkylisophthalamic acid (36). Conversion of this acid to an acid halide (37) is accomplished with thionyl chloride. (Hoey, U.S. Pat. No. 3,145,197).

The enamino ketone (43) is transformed with hydrazine hydrate into the tetrahydroindazole (44), which after acid hydrolysis provides 4,5,6,7-tetrahydro-2H-indazol-5-ylmethanamine (45). Masic, et al., Tetrahedron Lett 2000; 41 :5589-92.

Boc-D-cyclohexylglycine-proline (48) is prepared by standard amino acid coupling of Boc-D-cyclohexylglycine (46) with proline methyl ester (47), followed by ester hydrolysis. Standard amino acid coupling of Boc-D-cyclohexylglycine-proline with the amine (45), followed by removal of the Boc protecting group, produces the free amine (49). Tucker et al., J Med Chem 1998; 41:3210-19.

The acid chloride (37) is condensed with the amine (49), followed by the removal of hydroxyl protecting groups results in the formation of Compound VI.

### G. Example 7

### Acute Mouse Toxicity Assay

A group of female mice are dosed with iodine-containing anticoagulant contrast media at each dose level, and one group of female mice is dosed with vehicle via intravenous injection (caudal vein). The dose levels are 15, 13, 10, and 8 g Iodine/kg to estimate a median lethal dose, LD₅₀.

Observations for clinical signs and body weights are measured pretest and during the course of the study. Three animals per group are euthanized at two hours after dosing and the remaining animals euthanized five days after dosing. All animals receive a complete macroscopic examination. Tissues and organs are examined for evidence of hemorrhaging, clotting and other pathology. The following organs are collected from each animal for microscopic examination: brain, liver, lung, kidney, and skin (subcutaneous and adventitia). All animals dying spontaneously also receive a macroscopic evaluation. Because this is a lethality study, no animals will be sacrificed in extremis. The anticoagulant contrast agents of the invention will be as or, preferably, less toxic than the current commercially available contrast agents.

### H. Example 8

### Formulations and Administration of Active Compound

320 mg Iodine/ml (840 mM of compound I) is pH adjusted to 7.4 to 8.8 with NaOH or HCl in an aqueous solution consisting of 10 mM tromethane (Tris), 0.10 mg/ml edetate calcium disodium, 19 mM NaCl, and 0.3 mM Calcium chloride, dihydrate. This formulation is used in the dose regimens described below.

Selective coronary arteriography with or without left ventriculography. The usual dose for left coronary is 2-14 mL (typically 8 mL) of the formulation described above, and the usual dose for right coronary arteriography is 1 to 10 mL (typically 5 mL) of the formulation described above. The doses may be repeated as necessary. Doses up to a total of 150 mL are suitable. For left ventriculography, the usual dose in a single injection is 35-45 mL (typically 45 mL) and repeated as necessary. The total dose for combined selective coronary arteriography and left ventriculography should not exceed 250 mL.

Peripheral arteriography. The usual single adult dose for aorto-iliac runoff studies is 20 to 80 mL (typically 45 mL). The usual single adult dose for the common iliac, the external iliac and the femoral arteries is 10-50 mL (typically 30 mL). These doses may be repeated as necessary. For the upper limb, the usual single adult dose is 20 mL (range 15-30 mL) repeated as necessary. The total procedural dose should not exceed 250 mL.

Aortography and selective visceral arteriography. The usual dose for injections into the aorta is 25 to 50 mL; the usual dose for injection into the celiac artery is 40 mL; the usual dose for injection into the superior mesenteric artery is 20 to 40 mL; the usual dose for injection into the inferior mesenteric artery is 8 to 15 mL. These doses may be repeated as necessary. The total dose should not exceed 250 mL.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. An anticoagulant contrast agent comprising:
an organic scaffolding moiety;
an organic anticoagulant moiety; and
an imaging moiety,
wherein said scaffolding moiety functionally links said anticoagulant moiety to said imaging moiety, and wherein if the anticoagulant is: 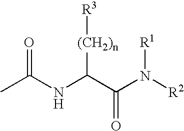
wherein n is two to six, R¹ and R² are independently hydrogen or alkyl or together form C4 to C8 alkylene, which is unsubstituted or substituted one to three times with lower alkyl or hydroxyl, and R³ is amino or guanidino, then the imaging moiety is not iodine.

2. The anticoagulant contrast agent of claim 1, wherein the anticoagulant moiety inhibits an enzyme associated with blood clotting.

3. The anticoagulant contrast agent of claim 2, wherein the enzyme is selected from the group consisting of thrombin, Factor Xa, Factor VIIa, and prothrombin.

4. The anticoagulant contrast agent of claim 2, wherein the anticoagulant moiety is a thrombin inhibitor, comprising a P1 component.

5. The anticoagulant contrast agent of claim 4, wherein the P1 component is an arginine mimetic.

6. The anticoagulant contrast agent of claim 5, wherein the arginine mimetic has the structure: 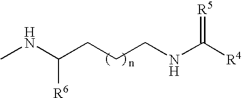
wherein
n is 0-3;
R⁴ is SR₄ or NHR⁷, wherein R is methyl or ethyl, and R⁷ is hydrogen, lower alkyl, halo, trihalomethyl, hydroxyl, alkoxy, amino, aryl, 5-membered heterocyclic ring, or 2- or 3-ring fused heterocyclic system;
R⁵ is O, S, or NR⁸, wherein R⁸ is hydrogen, lower alkyl, or hydroxyl; and
R⁶ is formyl, alkylcarbonyl, amino, dialkoxyboronyl, dialkylphosphatidyl.

7. The anticoagulant contrast agent of claim 6, wherein R¹ is methyl, phenyl, F, CF₃, propyl, hydroxyl, lower alkoxy, NHCH₃, thiazole, or benzothiazole, and R² is hydrogen, methyl, ethyl or hydroxyl.

8. The anticoagulant contrast agent of claim 5, wherein the arginine mimetic has the structure: 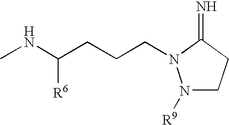
wherein,
R⁹ is hydrogen or lower alkyl; and
R⁶ is formyl, alkylcarbonyl, amino, dialkoxyboronyl, dialkylphosphatidyl.

9. The anticoagulant contrast agent of claim 5, wherein the arginine mimetic has the structure: 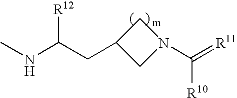
wherein
m is 2 or 3;
R¹⁰ is SR₄ or NHR¹³, wherein R is methyl or ethyl and R¹³ is hydrogen, lower alkyl, halo, trihalomethyl, hydroxyl, alkoxy, amino, a 5-membered heterocyclic ring or a 2- or 3-ring fused heterocyclic ring system;
R¹¹ is O, S, or NR¹⁴, wherein R¹⁴ is hydrogen, lower alkyl, or hydroxyl; and
R¹² is formyl, alkylcarbonyl, amino, dialkoxyboronyl, dialkylphosphatidyl.

10. The anticoagulant contrast agent of claim 4, wherein the P1 component is a lysine mimetic.

11. The anticoagulant contrast agent of claim 10, wherein the lysine mimetic has the structure: 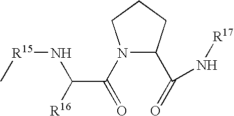
wherein,
R¹⁵ is a covalent bond or SO₂;
R¹⁶ is aryl; and
R¹⁷ is a 5- or 6-membered heterocyclic radical substituted with CH₂NH₂ or has the structure: 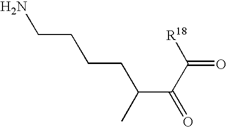
wherein R¹⁸ is amino or a 4- or 5-membered heterocyclic radical.

12. The anticoagulant contrast agent of claim 11, wherein R¹⁶ is phenyl.

13. The anticoagulant contrast agent of claim 4, wherein the thrombin inhibitor further comprises a P2 component.

14. The anticoagulant contrast agent of claim 13, wherein the thrombin inhibitor further comprises a P3 component.

15. The anticoagulant contrast agent of claim 3, wherein the anticoagulant moiety is a non-basic thrombin inhibitor.

16. The anticoagulant contrast agent of claim 1, wherein the imaging moiety is bromine or iodine.

17. The anticoagulant contrast agent of claim 16, wherein the scaffold comprises one to three benzyl rings.

18. The anticoagulant contrast agent of claim 17, wherein the scaffold moiety and imaging moiety together have the structure: 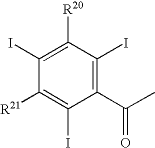
wherein, R²⁰ and R²¹ independently are amino or carbamoyl.

19. The anticoagulant contrast agent of claim 17, wherein the scaffold moiety and imaging moiety together have the structure: 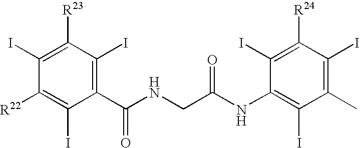
wherein, R²², R²³ and R²⁴ independently are amino, carbamoyl, or alkylcarbonyl.

20. The anticoagulant contrast agent of claim 16, wherein the scaffold comprises a cage compound.

21. The anticoagulant contrast agent of claim 20, wherein the cage compound is a dicarbon carborane cage, a fullerene, an adamantane, or a diamantine.

22. The anticoagulant contrast agent of claim 1, wherein the imaging moiety is an electron-dense heavy metal.

23. The anticoagulant contrast agent of claim 22, wherein the electron-dense heavy metal is selected from the group consisting of hafnium, tantalum, tungsten, rhenium, bismuth, and the lanthanide metals.

24. The anticoagulant contrast agent of claim 23, wherein the electron dense heavy metal is selected from the group consisting of tungsten, gadolinium, cerium and dysprosium.

25. The anticoagulant contrast agent of claim 23, wherein the scaffold is selected from the group consisting of a cage compound, and a metal chelator.

26. The anticoagulant contrast agent of claim 25, wherein the cage compound is a dicarbon carborane cage, a fullerene, an adamantane, or a diamantine.

27. The anticoagulant contrast agent of claim 25, wherein the scaffold is a derivative of H₃DO₃A-butrol.

28. A method of visualizing an internal structure comprising
(a) administering to a patient an amount of an anticoagulant contrast agent of claim 1; and
(b) exposing the internal structure to a diagnostic imaging procedure.

29. The method of claim 28, wherein the internal structure is a heart and the anticoagulant contrast agent is administered in conjunction with a catheterization procedure on the patient.

30. The method of claim 28, wherein the internal structure is a kidney or liver and the anticoagulant contrast agent is administered intravenously to the patient.

31. The method of claim 28, wherein the internal structure is a brain and the anticoagulant contrast agent is administered intrathecally to the patient.

32. The method of claim 28, where the diagnostic imaging procedure comprises an X-ray imaging procedure and the imaging moiety is iodine.

33. The method of claim 28, wherein the diagnostic imaging procedure comprises an MRI imaging procedure and the imaging moiety is an electron-dense heavy metal or iodine.

34. The method of claim 28, wherein the anticoagulant contrast agent is administered as a single bolus to the patient.

35. The method of claim 28, wherein the anticoagulant contrast agent is administered to the patient as several sequential doses.

36. A contrast media comprising an anticoagulant contrast agent as defined in claim 1 and a pharmaceutically acceptable carrier.

37. The contrast media of claim 36, wherein the pharmaceutically acceptable carrier is isotonic saline.

38. The contrast composition of claim 36, wherein the composition further comprises an antiemetic agent, a tranquilizer or a muscle relaxant.
